(19) United States
(12) Patent Application Publication
(10) Pub. No.: US 2023/0197200 A1
(43) Pub. Date: Jun. 22, 2023

(54) METHOD OF AND SYSTEM FOR PREDICTION OF VIRAL VARIANTS CHARACTERISTICS

(71) Applicant: nference, inc., Cambridge, MA (US)

(72) Inventors: Aiveliagaram J. VENKATAKRISHNAN, Cambridge, MA (US); Venkataramanan SOUNDARARAJAN, Andover, MA (US); Karthik MURUGADOSS, Brooklyn, NY (US); Bharathwaj RAGHUNATHAN, Mississauga (CA); Michiel NIESEN, Arlington, MA (US)

(21) Appl. No.: 18/087,337

(22) Filed: Dec. 22, 2022

Related U.S. Application Data
(60) Provisional application No. 63/293,066, filed on Dec. 22, 2021.

(51) Int. Cl. G16B 30/10; G16B 40/00

(52) U.S. Cl. CPC G16B 30/10; G16B 40/00

(57) ABSTRACT

In one aspect, a method includes receiving a plurality of biological sequence datasets, wherein each of the biological sequence datasets comprises a plurality of biological sequences; identifying a plurality of combinations of biological sequences, wherein each combination comprises one of the plurality of biological sequences from each of the biological datasets; and for each combination of biological sequences: generating a plurality of n-mers for each biological sequence of the combination using a sliding window with length n, comparing the plurality of n-mers for each biological sequence of the combination with the plurality of n-mers for the other biological sequences of the combination, identifying distinctive n-mers for each biological sequence of the combination which are not present among the plurality of n-mers for the other biological sequences of the combination, and determining a number of distinctive n-mers for at least one biological sequence of the combination.

### RELATED APPLICATIONS

This application claims priority to U.S. Provisional Application No. 63/293,066, filed Dec. 22, 2021, which is incorporated by reference herein in its entirety.

### FIELD OF THE INVENTION

The present disclosure relates to systems, methods, and computer readable media for prediction of viral variants and characteristics.

### BACKGROUND

As the COVID-19 pandemic has progressed over the last 2 years (December 2019-December 2021), highly transmissible and immuno-evasive SARS-CoV-2 variants have periodically emerged and dominated the global COVID-19 disease landscape. With over 5 million SARS-CoV-2 genomes available across sources such as in the Global Initiative on Sharing Avian Influenza Data (GISAID) resource, there is both an unprecedented opportunity and an acute need to decipher the molecular drivers facilitating the evolution of fitter SARS-CoV-2 variants.

The emergence of several SARS-CoV-2 Variants of Concern (VOCs: Alpha, Beta, Gamma, Delta, Omicron) over time has resulted in repeated surges of COVID-19 cases, hospitalizations, and deaths around the globe. Phylogenetic classification shows that these variants have evolved from common ancestors. The Pango lineages corresponding to these VOCs are as follows: Alpha—B.1.1.7 and Q lineages, Beta—B.1.351 and descendant lineages, Gamma—P.1 (a descendant of B.1.1.28) and descendant lineages, Delta—B.1.617.2 and AY lineages, Omicron—B.1.1.529 and BA lineages. Thus, all of these variants evolved from the B.1 lineage, while Alpha, Beta, and Gamma share B.1.1 as an additional parent lineage. However, these phylogenetic classifications do not intuitively describe the degree of distinctiveness between VOCs nor do they provide concrete insights into the genomic properties of each variant.

A new SARS-CoV-2 variant with a highly mutated spike protein was first reported from South Africa in November, 2021. This strain has since been denoted as the Omicron variant (WHO nomenclature) and B.1.1.529 (PANGO lineage). The rapid assessment of the variant by The Technical Advisory Group on SARS-CoV-2 Virus Evolution and classification of Omicron as a variant of concern by the WHO within 48 hours has facilitated timely epidemiological surveillance. Since the initial discovery of Omicron, the variant has already been detected in over 80 countries across six continents and has now become the dominant strain in circulation.

Comparison of Omicron variant with previous SARS-CoV-2 variants highlights the presence of novel mutations within the SARS-CoV-2 Spike protein, but a more complete understanding of the sequence diversity of the Omicron variant is essential to determine its evolutionary path and how it will shape the future trajectory of the COVID-19 pandemic. SARS-CoV-2, like other viruses, evolves via the introduction of mutations in its genome. In some cases, these mutations yield changes in the amino acid sequence of viral proteins. Such mutations can then be positively or negatively selected depending on their impact on various aspects of viral fitness, including transmissibility (e.g. ability to infect and/or replicate in host cells) and immune evasion (e.g. ability to avoid binding by host-derived neutralizing antibodies). It is clear from global data sharing events, particularly GISAID, that mutations in several regions such as the receptor binding domain and N-terminal domain of the Spike glycoprotein contribute to improved viral fitness. However, while much attention has been paid to the consequence of individual mutations at the amino acid level, there has been less focus on how SARS-CoV-2 explores the possibilities of diversifying its language at the level of nucleotide sequences. A detailed account of the mechanism through which the omicron strain emerged is critical to ongoing viral surveillance, vaccine design, and global vaccination strategy.

### BRIEF SUMMARY OF THE EMBODIMENTS

In one aspect, a method includes receiving a plurality of biological sequence datasets, wherein each of the biological sequence datasets includes a plurality of biological sequences; identifying a plurality of combinations of biological sequences, wherein each combination includes one of the plurality of biological sequences from each of the biological datasets; and for each combination of biological sequences: generating a plurality of n-mers for each biological sequence of the combination using a sliding window with length n, comparing the plurality of n-mers for each biological sequence of the combination with the plurality of n-mers for the other biological sequences of the combination, and identifying distinctive n-mers for each biological sequence of the combination which are not present among the plurality of n-mers for the other biological sequences of the combination, and determining a number of distinctive n-mers for at least one biological sequence of the combination.

In some embodiments, the plurality of biological sequence datasets include a plurality of genome datasets and each of the plurality of genome datasets includes a plurality of polynucleotide sequences.

In some embodiments, the plurality of biological sequence datasets include a plurality of protein sequence datasets and each of the plurality of protein sequence datasets includes a plurality of protein sequences.

In some embodiments, each biological sequence of the combination is aligned to a reference sequence before generating a plurality of n-mers for each biological sequence of the combination and comparing the plurality of n-mers for each biological sequence of the combination includes comparing n-mers at the same position of each biological sequence of the combination.

In some embodiments, comparing the plurality of n-mers for each biological sequence of the combination includes comparing n-mers regardless of the position of each n-mer in each biological sequence of the combination

In some embodiments, determining the number of distinctive n-mers for at least one biological sequence of the combination includes determining a number of distinctive n-mers for each biological sequence of the combination; and wherein the method further includes: generating a distribution for each of the plurality of biological sequence datasets of the number of distinctive n-mers for each biological sequence of each of the combinations.

In some embodiments, a divergence between each distribution is calculated using one or more of Cohen's D and J-S Divergence.

In some embodiments, each combination of biological sequences includes a first biological sequence from a first biological sequence dataset and one of the plurality of biological sequences from a second biological sequence dataset, and wherein determining the number of distinctive n-mers for at least one biological sequence of the combination includes determining a number of distinctive n-mers for the first biological sequence that are not present among the plurality of n-mers for the biological sequence from the second biological sequence dataset of the combination; and wherein the method further includes: determining a sequence distinctiveness for the first biological sequence by summing the number of distinctive n-mers from all combinations and dividing by the number of combinations.

In some embodiments, the plurality of biological sequence datasets include a first biological sequence dataset and a second biological sequence dataset, and the method further includes calculating a sequence distinctiveness for one or more biological sequences of the first biological sequence dataset relative to the second biological sequence dataset.

In some embodiments, one of the plurality of biological sequence datasets is a new viral variant sequence dataset.

In some embodiments, n is 9.

In some embodiments, n is 1.

In some embodiments, n is 9-30.

In some embodiments, n is 3-10.

In some embodiments, the method further includes identifying common n-mers that are present among the plurality of n-mers for two or more biological sequences in a combination of biological sequences.

In some embodiments, each of the plurality of biological sequence datasets is from a different time window.

In some embodiments, each of the plurality of biological sequence datasets is from a different geographical location.

In some embodiments, each of the plurality of biological sequence datasets is from a different variant.

In some embodiments, the plurality of biological sequence datasets includes a biological sequence dataset from an infectious agent and a biological sequence datasets from a host organism of the infectious agent.

In some embodiments, generating the plurality of n-mers includes generating a plurality of n-mers from only a functionally relevant portion of the plurality of biological sequences.

In some embodiments, the method further includes using the number of distinctive n-mers or a parameter derived therefrom to predict changes in prevalence.

In one aspect, a system includes a non-transitory memory; and one or more hardware processors configured to read instructions from the non-transitory memory that, when executed cause one or more of the hardware processors to perform operations including: receiving a plurality of biological sequence datasets, wherein each of the biological sequence datasets includes a plurality of biological sequences; identifying a plurality of combinations of biological sequences, wherein each combination includes one of the plurality of biological sequences from each of the biological datasets; and for each combination of biological sequences: generating a plurality of n-mers for each biological sequence of the combination using a sliding window with length n, comparing the plurality of n-mers for each biological sequence of the combination with the plurality of n-mers for the other biological sequences of the combination, identifying distinctive n-mers for each biological sequence of the combination which are not present among the plurality of n-mers for the other biological sequences of the combination, and determining a number of distinctive n-mers for at least one biological sequence of the combination.

In some embodiments, determining the number of distinctive n-mers for at least one biological sequence of the combination includes determining a number of distinctive n-mers for each biological sequence of the combination; and wherein the operations further include: generating a distribution for each of the plurality of biological sequence datasets of the number of distinctive n-mers for each biological sequence of each of the combinations.

In some embodiments, each combination of biological sequences includes a first biological sequence from a first biological sequence dataset and one of the plurality of biological sequences from a second biological sequence dataset, and wherein determining the number of distinctive n-mers for at least one biological sequence of the combination includes determining a number of distinctive n-mers for the first biological sequence that are not present among the plurality of n-mers for the biological sequence from the second biological sequence dataset of the combination; and wherein the operations further include: determining a sequence distinctiveness for the first biological sequence by summing the number of distinctive n-mers from all combinations and dividing by the number of combinations.

In some embodiments, n is 9-30.

In some embodiments, n is 3-10.

In some embodiments, the operations further include identifying common n-mers that are present among the plurality of n-mers for two or more biological sequences in a combination of biological sequences.

In some embodiments, each of the plurality of biological sequence datasets is from a different time window.

In some embodiments, each of the plurality of biological sequence datasets is from a different geographical location.

In some embodiments, each of the plurality of biological sequence datasets is from a different variant.

In one aspect, a non-transitory computer-readable medium stores instructions that, when executed by one or more hardware processors, cause the one or more hardware processors to perform operations including: receiving a plurality of biological sequence datasets, wherein each of the biological sequence datasets includes a plurality of biological sequences; identifying a plurality of combinations of biological sequences, wherein each combination includes one of the plurality of biological sequences from each of the biological datasets; and for each combination of biological sequences: generating a plurality of n-mers for each biological sequence of the combination using a sliding window with length n, comparing the plurality of n-mers for each biological sequence of the combination with the plurality of n-mers for the other biological sequences of the combination, identifying distinctive n-mers for each biological sequence of the combination which are not present among the plurality of n-mers for the other biological sequences of the combination, and determining a number of distinctive n-mers for at least one biological sequence of the combination.

In some embodiments, determining the number of distinctive n-mers for at least one biological sequence of the combination includes determining a number of distinctive n-mers for each biological sequence of the combination; and wherein the operations further include: generating a distribution for each of the plurality of biological sequence datasets of the number of distinctive n-mers for each biological sequence of each of the combinations.

In some embodiments, each combination of biological sequences includes a first biological sequence from a first biological sequence dataset and one of the plurality of biological sequences from a second biological sequence dataset, and wherein determining the number of distinctive n-mers for at least one biological sequence of the combination includes determining a number of distinctive n-mers for the first biological sequence that are not present among the plurality of n-mers for the biological sequence from the second biological sequence dataset of the combination; and wherein the operations further include: determining a sequence distinctiveness for the first biological sequence by summing the number of distinctive n-mers from all combinations and dividing by the number of combinations.

In some embodiments, n is 9-30.

In some embodiments, n is 3-10.

In some embodiments, the operations further include identifying common n-mers that are present among the plurality of n-mers for two or more biological sequences in a combination of biological sequences.

In some embodiments, each of the plurality of biological sequence datasets is from a different time window.

In some embodiments, each of the plurality of biological sequence datasets is from a different geographical location.

In some embodiments, each of the plurality of biological sequence datasets is from a different variant.

Any one of the embodiments disclosed herein may be properly combined with any other embodiment disclosed herein. The combination of any one of the embodiments disclosed herein with any other embodiments disclosed herein is expressly contemplated.

### BRIEF SUMMARY OF THE DRAWINGS

The objects and advantages will be apparent upon consideration of the following detailed description, taken in conjunction with the accompanying drawings, in which like reference characters refer to like parts throughout, and in which:

FIG. 1 shows an illustrative embodiment for analyzing distinctive n-mers in biological sequences, according to certain embodiments.

FIG. 2 shows an illustrative embodiment for analyzing distinctive n-mers in biological sequences using an alignment-free genome-based method, according to certain embodiments.

FIG. 3 shows an illustrative embodiment for analyzing distinctive n-mers in biological sequences using an aligned protein-based method, according to certain embodiments.

FIG. 4A shows density plots of 9-mer sequence diversity, as measured by the number of distinctive polynucleotide sequences in SARS-CoV2 variants of concern, according to certain embodiments.

FIG. 4B shows density plots of 15-mer sequence diversity, as measured by the number of distinctive polynucleotide sequences in SARS-CoV2 variants of concern, according to certain embodiments.

FIG. 4C shows geographic distribution of Alpha, Beta, and Gamma variants based on sequences deposited in GISAID through 14 Dec. 2021, according to certain embodiments.

FIG. 5A shows a Venn diagram showing the number of distinctive and shared 9-mer sequences for SARS-CoV2 variants of concern, according to certain embodiments.

FIG. 5B shows heatmaps of Cohen's D values from pairwise comparisons of the distribution of distinctive 9-mer sequences between SARS-CoV2 variants of concern, according to certain embodiments.

FIG. 5C shows heatmaps of Jensen-Shannon divergence values from pairwise comparisons of the distribution of distinctive 9-mer sequences between SARS-CoV2 variants of concern, according to certain embodiments.

FIG. 6A shows density plots of the distribution of the number of mutations observed in sequences deposited in the GISAID database, according to certain embodiments.

FIG. 6B shows heatmaps of Cohen's D values from pairwise comparisons of the distribution of number of mutations between SARS-CoV2 variants of concern, according to certain embodiments.

FIG. 6C shows heatmaps of Jensen-Shannon Divergence values from pairwise comparisons of the distribution of number of mutations between SARS-CoV2 variants of concern, according to certain embodiments.

FIG. 7A shows heatmaps of the mean phylogenetic distance between SARS-CoV2 variants of concern calculated using the Tajima-Nei method, according to certain embodiments.

FIG. 7B shows heatmaps of the mean phylogenetic distance between SARS-CoV2 variants of concern calculated using the Tamura method, according to certain embodiments.

FIG. 7C shows heatmaps of the mean phylogenetic distance between SARS-CoV2 variants of concern calculated using the Jukes Cantor method, according to certain embodiments.

FIG. 7D shows heatmaps of the mean phylogenetic distance between SARS-CoV2 variants of concern calculated using the Kimura method, according to certain embodiments.

FIG. 8 shows the Cohen's D values of the distinctive n-mer distributions of the Alpha, Beta, Gamma, Delta, and Omicron variants against the Wuhan Strain for different n-mer lengths, according to certain embodiments.

FIG. 9 shows density plots of 9-mer sequence diversity, as measured by the number of distinctive polynucleotide sequences in SARS-CoV2 variants of interest, according to certain embodiments.

FIG. 10 shows a global metric for 9-mer distinctiveness plotted against lineage emergence date for all GISAID lineages, according to certain embodiments

FIG. 11A shows the 9-mer distinctiveness of the Wuhan strain, Alpha, Beta, and Gamma variants before June 2021, according to certain embodiments.

FIG. 11B shows the 9-mer distinctiveness of the Wuhan strain, Alpha, Beta, Gamma, and Delta variants before November 2021, according to certain embodiments.

FIG. 12A shows a probability density function of the number of genes per HCoV strain, according to certain embodiments.

FIG. 12B shows a probability function of total sequence length per HCoV strain, according to certain embodiments.

FIG. 12C shows a probability density function of the number of distinctive 9-mers per HCoV strain, according to certain embodiments.

FIG. 12D shows a probability function of the number of distinctive 9-mers for HCoV compared to variants of concern, according to certain embodiments.

FIG. 13 shows density plots of 9-mer sequence diversity for 9-mers in the spike protein, as measured by the number of distinctive polynucleotide sequences in SARS-CoV2 variants of interest, according to certain embodiments.

FIG. 14 shows density plots of 9-mer sequence diversity for the Wuhan strain, Delta, and Omicron, with Delta split into two subgroups based on time, according to certain embodiments.

FIG. 15A shows the probability density functions of distinctive 9-mers for Wuhan, Alpha, Beta, Gamma, Delta, and Omicron, where the Delta variant sequences are from July-August 2021, according to certain embodiments.

FIG. 15B shows the probability density functions of distinctive 9-mers for Wuhan, Alpha, Beta, Gamma, Delta, and Omicron, where the Delta variant sequences are from November-December 2021, according to certain embodiments.

FIG. 15C shows the probability density functions of distinctive 9-mers for Wuhan, Alpha, Beta, Gamma, Delta, and Omicron, where the Delta variant sequences are from April-May-June 2021, according to certain embodiments.

FIG. 15D shows the probability density functions of distinctive 9-mers for Wuhan, Alpha, Beta, Gamma, Delta, and Omicron, where the Delta variant sequences are from December 2021, according to certain embodiments.

FIGS. 16A-16D show distributions of distinctive n-mers in a user interface, according to certain embodiments.

FIGS. 17A-17F show Venn diagrams of n-mers in a user interface, according to certain embodiments.

FIG. 18A shows a schematic of calculation of mutational load for a protein sequence, according to certain embodiments.

FIG. 18B shows a schematic of calculation of sequence Distinctiveness for a protein sequence, according to certain embodiments.

FIG. 19A shows the prevalence of different variants in India as a function of time, according to certain embodiments.

FIG. 19B shows mutational load for different variants in India over time, according to certain embodiments.

FIG. 19C shows Distinctiveness for different variants in India over time, according to certain embodiments.

FIG. 19D shows mutational load for Delta sequences compared to other sequences in India in January 2021, according to certain embodiments.

FIG. 19E shows Distinctiveness of Delta compared to other sequences in Indian in January 2021, according to certain embodiments.

FIG. 19F shows the prevalence of different variants in Brazil as a function of time, according to certain embodiments.

FIG. 19G shows mutational load for different variants in Brazil over time, according to certain embodiments.

FIG. 19H shows Distinctiveness for different variants in Brazil over time, according to certain embodiments.

FIG. 19I shows mutational load for Delta sequences compared to other sequences in Brazil in June 2021, according to certain embodiments.

FIG. 19J shows Distinctiveness of Delta compared to other sequences in Brazil in June 2021, according to certain embodiments.

FIGS. 20A-20B show the Distinctiveness of the Alpha variant compared to other sequences in the United Kingdom in October 2020, according to certain embodiments.

FIGS. 20C-20D show the Distinctiveness of the Beta variant in South Africa in October 2020, according to certain embodiments.

FIGS. 20E-20F show the Distinctiveness of the Gamma variant in Brazil in October 2020, according to certain embodiments.

FIGS. 20G-20H show the Distinctiveness of the Delta variant in India in January 2021, according to certain embodiments.

FIGS. 201-20J show the Distinctiveness of the Omicron variant in South Africa in November 2021, according to certain embodiments.

FIGS. 21A-21B show the mutational load of the Alpha variant compared to other sequences in the United Kingdom in October 2020, according to certain embodiments.

FIGS. 21C-21D show the mutational load the Beta variant in South Africa in October 2020, according to certain embodiments.

FIGS. 21E-21F show the mutational load of the Gamma variant in Brazil in October 2020, according to certain embodiments.

FIGS. 21G-21H show the mutational load of the Delta variant in India in January 2021, according to certain embodiments.

FIGS. 21I-21J show the mutational load the Omicron variant in South Africa in November 2021, according to certain embodiments

FIG. 22A shows a Venn diagram comparing the positions that contribute to Mutational load in India and Brazil, according to certain embodiments.

FIG. 22B shows a Venn diagram comparing the positions that contribute to high Distinctiveness in India and Brazil, according to certain embodiments.

FIG. 22C shows positions of a protein that are distinctive in both India and Brazil, according to certain embodiments.

FIG. 22D shows positions of a protein that are distinctive in Brazil only, according to certain embodiments.

FIG. 23A shows the mutational load at different positions along the spike protein for Delta sequences in India during January 2021, according to certain embodiments.

FIG. 23B shows distinctiveness at different positions along the spike protein for Delta sequences in India during January 2021, according to certain embodiments.

FIG. 23C shows the mutational load at different positions along the spike protein for Delta sequences in Brazil during June 2021, according to certain embodiments.

FIG. 23D shows the distinctiveness at different positions along the spike protein for Delta sequences in Brazil during June 2021, according to certain embodiments.

FIG. 24A shows the change in prevalence as a function of distinctiveness of each lineage relative to the average distinctiveness of all sequences collected in the region at the same time, according to certain embodiments.

FIG. 24B shows the receiver operator curve (ROC) for predicting an increase in prevalence, according to certain embodiments.

FIG. 25A shows the correlation between change in prevalence of a lineage, from the current time to 56 days in the future, without any filtering of time periods, and the distinctiveness of each lineage relative to the average distinctiveness, according to certain embodiments.

FIG. 25B shows the ROC for predicting an increase in prevalence, according to certain embodiments.

FIG. 26A shows the AUC for the prediction of lineages with increase in prevalence 56 days in the future from the average Distinctiveness of sequences belonging to that lineage, according to certain embodiments.

FIG. 26B shows optimal threshold values for a positive prediction, according to certain embodiments.

FIG. 27 shows Distinctiveness of only Spike protein positions compared to Distinctiveness of a full proteome, according to certain embodiments.

FIG. 28 shows analysis of Distinctiveness at the US state level, according to certain embodiments.

### DETAILED DESCRIPTION

While mutation load and codon frequency characterization methods focus on individual nucleotides and in-frame 3-mer fragments respectively, the methods and systems described herein are based on the observation that recently evolved, highly transmissible variants of concern (VOCs) such as Omicron and Delta have a significantly larger proportion of distinctive long polynucleotide fragments (e.g., 9-30-mer or more) or polypeptide fragments (e.g., 3-10-mer or more). Distinctive fragments were also observed at the proteome level for VOCs.

Based on this observation, a method quantifies the fragments of biological sequences (e.g., polynucleotides or proteins) in lineages of infectious agents. In some embodiments, this method quantifies biological sequences of viral (e.g., SARS-CoV-2) lineages. This method can be used to compare biological sequences from different datasets corresponding to different time windows, geographic locations, variants, species, or combinations thereof. In some embodiments, this method can distinguish between the viral (e.g., SARS-CoV-2) variants of concern and rank-order them by date of emergence more effectively than other methods. Overall, the methods described herein demonstrate the utility of comparing biological sequence datasets based on their distinctive fragments (e.g., distinctive long polynucleotide fragments or amino acid fragments), in order to infer phylogenetic information and to gain insight in potential fitness of new variants.

The methods and systems described herein can be used to compare any biological sequence with existing reference or comparative sequences. For example, an emerging strain or variant can be compared with existing strains or variants of a virus to predict whether the emerging strain or variant is likely to be a highly transmissive or fit variant. For example, an emerging strain or variant can be compared with previously collected sequences. When new strains emerge is an open question, and the methods disclosed herein can provide a means of predicting emerging strains. For example, these methods can predict which emerging strains or variants are likely to become dominant. Alternatively, the methods and systems described herein can be used to compare across different viruses, e.g. SARS-CoV-2, influenza, seasonal coronaviruses, and adenoviruses. In this way, these methods can serve as part of a pan-respiratory monitoring platform.

In some embodiments, changes in a viral genome or proteome indicate whether there is likely to be reduced immunity to new strains of the virus. For example, if a new strain of a virus is similar to a prior strain of a virus, then past exposure to the prior strain, e.g., via infection or vaccination, may provide immunity against a new strain. Alternatively, if a new strain is sufficiently different from the prior strain, immunity based on past exposure to the prior strain may be diminished. The methods and systems described herein can be applied, for example, to develop effective diagnostic tests and predict which available vaccines (e.g., mRNA vaccines, adenoviral vaccines, or booster shots) provide immunity against a new strain. Similarly, comparison across different viruses can be used to determine whether exposure to one virus provides any immunity against another virus.

Disclosed herein are systems and methods for comparing different datasets of biological sequences. For example, a biological sequence can be compared to one or more comparative sequences to identify distinctive n-mers. Using these systems and methods disclosed herein, combinations of biological sequences from different datasets can be compared by identifying distinctive n-mers for each biological sequence. Here, distinctive n-mers are n-mer sequences that occur within a biological sequence or biological sequence dataset that are not present in the other a biological sequence or biological sequence dataset being evaluated, where n is the number of monomers an n-mer sequence. For a first biological sequence, all possible n-mers can be determined and compared with the n-mers for a second biological sequence from a different dataset (e.g., a comparative biological sequence). If an n-mer is present in the first biological sequence but not the second biological sequence (comparative biological sequence), that n-mer is distinctive. In some embodiments, sequences are aligned with a reference sequence before comparing n-mers. In other embodiments, sequences are not aligned before comparing n-mers. Disclosed herein are various methods of comparing biological sequences and identifying distinctive n-mers. In some embodiments, these methods can be used to identify whether emerging strain or variant is likely to be highly transmissive or fit.

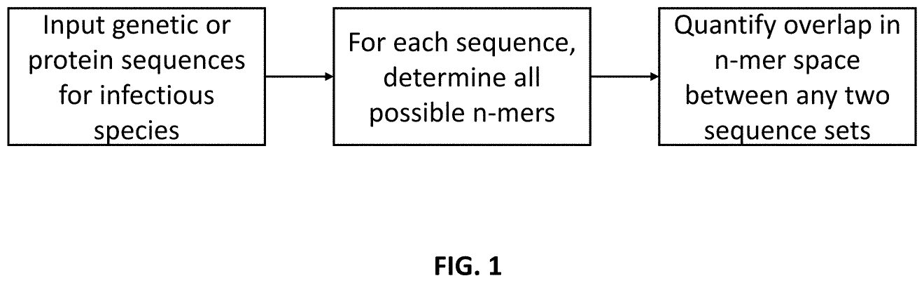

FIG. 1 shows an illustrative embodiment for analyzing distinctive n-mers. As shown in FIG. 1, a plurality biological sequences (e.g., genetic or protein sequences for an infectious species) can be input. These biological sequences can be grouped into different data sets (e.g., based on time windows, geographic location, species, or strain). Then, for each sequence all possible n-mers can be determined. In some embodiments, n-mers can be identified using a sliding window with length n. Then, for any two biological sequences or for any two sets of sequences, the overlap in n-mer space can be quantified. For example, for a given biological sequence, the number of distinctive n-mers relative to one or more comparative biological sequences can be determined. The number of distinctive n-mers can be used, for example, to generate a distribution or histogram of distinctive n-mers or to calculate a distinctiveness metric.

A biological sequence can include any one-dimensional ordering of monomers. In some embodiments, a biological sequence is a polynucleotide sequence or genome sequence. In a polynucleotide or genome sequence, a monomer is a nucleotide (e.g. a deoxyribonucleotide or ribonucleotide). In some embodiments, a biological sequence is an amino acid sequence, a protein sequence, or polypeptide sequence. In an amino acid, protein sequence or polypeptide sequence, a monomer is an amino acid. In some embodiments, the methods and systems disclosed herein can be used to analyze multiple different sequences for an organism, e.g., multiple chromosomes in a genome or multiple proteins in a proteome.

In some embodiments, the biological sequence is derived from an infectious agent, e.g., a virus or a bacteria. In some embodiments, the biological sequence is derived from a host organism of an infectious agent. In some embodiments, the methods and systems disclosed herein can be used to compare biological sequences of an infectious agent with biological sequences of a host organism of that infectious agent to identify overlap or distinctive n-mers between host sequences and infectious agent sequences. In some embodiments, comparing biological sequences of an infectious agent with biological sequences of a host organism can provide information about autoimmune response

In some embodiments, the length of n-mers is 3, 6, 9, 12, 15, 18, 21, 24, 30, 45, 60, 75, 120, 240, or in any range bounded by any value disclosed herein. In some embodiments, the length of n-mers is 9. In some embodiments, the length of n-mers is 9-30, 9-24, 18-24, or 18-30. In some embodiments, the length of n-mers is 3. In some embodiments, the length of n-mers is 3-10. In some embodiments, the length of n-mers n is 9-30 and the biological sequences are polynucleotide sequences. In some embodiments, the length of n-mers is 3, and the biological sequences are aligned polynucleotide sequences. In some embodiments, the length of n-mers is 3-10, and the biological sequences are unaligned protein sequences. In some embodiments, the length of n-mers is 1, and the biological sequences are aligned protein sequences.

The methods and systems disclosed herein can be used to analyze unaligned or aligned biological sequences. In an unaligned or alignment free method, an n-mer of a biological sequence is distinctive relative to one or more comparative biological sequences if it is different from all n-mers in the comparative biological sequence(s), regardless of the position of each n-mer. In an aligned method, each sequence is aligned to a reference biological sequence (e.g., an ancestral sequence) before comparing n-mers. In such an aligned method, an n-mer of a biological sequence is distinctive relative to one or more comparative biological sequences if it is different from the n-mer at the same position of the comparative sequence(s).

In some embodiments, the biological sequences can be grouped into different datasets to allow comparison of n-mers among or between different groups or to identify distinctive n-mers relative to different groups. In some embodiments, datasets can be grouped based on time. For example, a dataset of biological sequences from a time window can be compared with a dataset that includes biological sequences from all prior time points. In another example, a dataset of biological sequences from a time window can be compared with one or more datasets from one or more earlier time windows. Non limiting examples of time windows include periods of days, weeks, or months. In some embodiments, a time window is 6 months in length. In some embodiments, a time window is selected based on the immune memory for an infectious agent. For example, in the context of SARS-CoV, a 6-month window is relevant to immune memory. In some embodiments, datasets can be grouped based on geographic location. For example, datasets can be grouped based on continent, region, country, state, or municipality. In some embodiments, datasets can be grouped based on variant or strain. For example, datasets with biological sequences from viruses can be grouped by variant of concern (VOC) or variant of interest (VOI). For example, a variant can be compared to one or more other variants. In another example, a variant can be compared to all other contemporary variants. In some embodiments, datasets can be grouped based on species. In some embodiments, datasets can be grouped based on combinations of time windows, geographic locations, strain, variant, and species. Such combinations can allow identification of a variant in a particular region or at a particular time. For example, biological sequences from a first variant in a first location during a first time window can be compared with biological sequences from a second variant in the same location at the same time. In another example, biological sequences from a first variant at a first time in a first location can be compared to biological sequences from the same variant in the same location but at a second time. In some embodiments, grouping datasets using any of the categories described herein can be used to identify emerging variants with increased distinctiveness.

In some embodiments, analysis of distinctive n-mers is limited to a particular portion of a biological sequence, for example a portion of the biological sequence that is functionally relevant. Such analysis can be used to determine whether n-mer distinctiveness is increased in certain portions of a biological sequence. Examples of functionally relevant biological sequences include portions of a biological sequence corresponding to a spike protein of a virus, a biological sequence corresponding to a binding site of a protein, a biological sequence corresponding to an epitope of an antigen, or a biological sequence corresponding to antibody accessible or exposed portions of a protein. Non-limiting examples of functionally relevant sequences include NTD and RBD of the SARS-CoV-2 spike protein. In some embodiments, increased distinctiveness in a functionally relevant portion of a genome can be predictive of increased prevalence, e.g., because a difference in a functionally relevant portion can provide increased fitness.

In some embodiments, the number of distinctive n-mers for one or more biological sequences can be used to quantitatively compare different biological sequence datasets. In some embodiments, parameters derived from the number of distinctive n-mers or positions of distinctive n-mers can be used to quantitatively compare distinctiveness of different biological sequence datasets. For example, the distributions of the number of distinctive n-mers for each group of biological sequence datasets can be compared. In another example, a metric or score can be used to quantify distinctiveness of a biological dataset relative to a comparative biological sequence dataset. For example, a n-mer distinctiveness metric can be calculated for a particular n or a sequence distinctiveness metric can be calculated for a particular sequence.

In some embodiments, a distribution of the probability density for the number of distinctive n-mers in each biological sequence dataset can be used to compare distinctiveness of different biological sequence datasets. In some embodiments, such distributions can be generated by identifying a plurality of combinations of biological sequences, each combination including a biological sequence from each of the biological sequence datasets. In some embodiments, each dataset corresponds to a different variant. In some embodiments, each data set corresponds to a different time window. In some embodiments, identifying combinations from different datasets allows sampling of biological sequences from each dataset without a need to analyze all sequences in all datasets. For each combination, the number of distinctive n-mers for each biological sequence can be determined by comparing the n-mers of that biological sequence to the n-mers for one or more of the other biological sequences in the combination. After sampling the plurality of combinations and determining the number of distinctive n-mers for each sequence, a probability distribution or density plot can be generated for each biological sequence dataset showing a histogram of the number of distinctive n-mers for each of the biological sequences of that biological sequence dataset. In some embodiments, probability distributions can be used to identify an emerging variant. For example, if a variant dataset has a bimodal or multimodal distribution of number of n-mers, that may indicate that the variant dataset could be split into at least two groups, with sequences having an increased number of distinctive n-mers belonging to an emerging variant. In some embodiments, distributions of distinctive n-mers provide greater resolution between variants than distributions of mutational load.

In some embodiments, a divergence can be used to quantitatively compare two distributions corresponding to two different biological sequence datasets. For example, the Cohen's D or J-S Divergence can be used to quantitatively compare pairs of different biological sequence datasets. In some embodiments, a significant divergence can be used to identify a variant of concern. In some embodiments divergence (e.g., Cohen's D or J-S Divergence) is a better predictor of a variant of concern than other metrics such as mutational load and phylogenetic distance.

In some embodiments, an n-mer distinctiveness metric, A*(1−B) can be used to quantify an n-mer-specific distinctiveness for a given biological sequence dataset or group. For a given biological sequence dataset, the following n-mer distinctiveness metric can be calculated

l=<A(l,n)*(1−B(l,n))>ₙ,

where A(l,n) is the fraction of sequences in dataset l that contain a specific n-mer, n, and B(l,n) is the fraction of sequences from all other comparative datasets other dataset l that contain n-mer n. The angular brackets indicate averaging over all n-mers that are reported for dataset l. In some embodiments, the n-mer distinctiveness metric can be analyzed over time or for different time windows. An n-mer distinctiveness metric can be used for an n-mer of any length and for aligned or unaligned sequences. As such, this metric can be used if aligned sequences are not available (e.g. because of poor sequencing or incomplete data) or if comparing sequences that cannot be readily aligned (e.g., comparing difference species, such as comparing a sequence of an infectious agent with a sequence of a host organism or comparing sequences of infectious agents of different species).

In some embodiments, a sequence Distinctiveness can be calculated. For a given sequence from a first dataset, that sequence can be compared to a plurality of sequences from a second, comparative dataset. In some embodiments, the first dataset is from a first time window, and the second dataset is from a second time window (e.g., all earlier sequences). In some embodiments, the first data set is from a first variant, and the second dataset is from one or more other variants. In some embodiments, a sequence distinctiveness for a biological sequence can be calculated by summing the number of distinctive n-mers compared to all sequences from a second, comparative dataset and dividing by the number of sequences in the comparative dataset. In some embodiments, a sequence Distinctiveness can be calculated for aligned biological sequences. In some embodiments, a sequence Distinctiveness can be calculated for unaligned sequences. In some embodiments, the sequence Distinctiveness is normalized by the number of sequences in the number of sequences in the comparative dataset. In some embodiments, the sequence Distinctiveness is normalized by the number of distinctive n-mers in the sequence.

For aligned sequences, the sequence Distinctiveness can be calculated using the following formula:

$$D ( s ) = \frac{1}{{N}_{c}} {\sum }_{{s}^{′} ∈ comparative dataset} {\sum }_{p = 1}^{\# monomers - n + 1} 1 - δ ( s ( p ) - {s}^{′} ( p ) )$$

Where N_{c} is the number of sequences in the second, comparative dataset, s′ is one specific sequence from the comparative dataset, the outer sum is over all sequences in the second comparative dataset, the inner sum is over all pairwise aligned n-mer positions, and δ(s(p)−s′(p)) evaluates to 1 if sequence s and s′ have the same n-mer at position p and 0 otherwise. In some embodiments, the positions are determined relative to a reference sequence. In some embodiments, the sequence Distinctiveness can be calculated for a plurality of sequences from a biological sequence dataset (e.g., sequences from a particular time window, geographic location, or variant). In some embodiments, the sequence Distinctiveness can also be normalized by the number of n-mers in the sequence s.

For unaligned sequences, the sequence Distinctiveness can be calculated using the following formula:

$$D ( s ) = \frac{1}{{N}_{c}} {\sum }_{{s}^{′} ∈ comparative dataset} {\sum }_{n ∈ s} 1 - δ ( {s}^{′} ( n ) )$$

Where N_{c} is the number of sequences in the second, comparative dataset, the outer sum is over all sequences in the second comparative dataset, s′ is one specific sequence from the comparative dataset, the inner sum is over all n-mers n in sequence s, and δ(s′(n)) evaluates to 1 if sequence s′ includes n-mer n and 0 otherwise. In some embodiments, this sequence Distinctiveness can also be normalized by the number of n-mers in the sequence s.

In some embodiments, the sequence Distinctiveness can be a better predictor of a variant of concern than other metrics such as mutational load. One benefit of sequence Distinctiveness over mutational load, even for short n-mers such as 1-mers, is that mutational load compares each sequence only to a single reference sequence, often an ancestral sequence or wild type sequence. In contrast, sequence Distinctiveness can incorporate comparisons to a large number of sequences in a comparative dataset, and that comparative dataset can be selected, for example to include sequences from a particular time window, geographic location, or set of variants. In this way, sequence distinctiveness can provide a more dynamic or flexible comparison than mutational load. Additionally, in some embodiments, contributions of sequences in a comparative dataset to sequence Distinctiveness can be weighted, e.g., by recency or prevalence of the sequences in the comparative data set. For example, the weight of more prevalent sequences can be increased. In one example, in 2022, there would be a large number of Omicron sequences in a contemporary comparative dataset, so Omicron will be weighted more heavily. In another example, a weight can be introduced for variants if the prevalence of each variant in the dataset is not representative of the prevalence in the population at all. For example, the contribution of more recent sequences can be reduced. In one example, the following weight can be applied to a sequence based on collection date:

e^{-At}

where A is a coefficient and t is the time (e.g., days) since the collection date.

In some embodiments, a position Distinctiveness can be calculated for each n-mer in a sequence. For a given sequence from a first dataset, the n-mer at a position can be compared to the corresponding n-mer at the same position in a plurality of sequences from a second, aligned, comparative dataset. A position Distinctiveness can be used to identify which positions in a sequence contribute to distinctiveness. In some embodiments, the first dataset is from a first time window, and the second dataset is from a second time window (e.g., all earlier sequences). In some embodiments, the first data set is from a first variant, and the second dataset is from one or more other variants. For aligned sequences, the position Distinctiveness can be calculated using the following formula:

$$D ( p ) = \frac{1}{{N}_{c}} {\sum }_{{s}^{′} ∈ comparative dataset} 1 - δ ( s ( p ) - {s}^{′} ( p ) )$$

Where N_{c} is the number of sequences in the second, comparative dataset, s′ is one specific sequence from the comparative dataset, and δ(s(p)−s′(p)) evaluates to 1 if sequence s and s′ have the same n-mer at position p and 0 otherwise. In some embodiments, the positions are determined relative to a reference sequence. In some embodiments, this sequence Distinctiveness can also be normalized by the number of n-mers in the sequence s.

In some embodiments, the methods and systems disclosed herein include identifying common n-mers. In some embodiments, common n-mers are present among two or more of the biological sequences from different datasets. In some embodiments, common n-mers are present among two or more different biological datasets. In some embodiments, common n-mers are present among all biological sequences being compared. In some embodiments, common n-mers are present among all biological sequence datasets being compared. In some embodiments common n-mers can be indicative of conserved or stable sequences. In some embodiments, common n-mers can be shown using a Venn Diagram.

In some embodiments, identifying distinctive n-mers and quantification of distinctiveness can be used to predict emergence of new variants. In some embodiments, the number of distinctive n-mers or any parameter derived therefrom can be used to make such predictions. For example, the number of distinctive n-mers or any parameter derived therefrom can be used to predict future changes in prevalence. For example, the number of distinctive n-mers or any parameter derived therefrom can be used to predict infectious disease outcomes. Non-limiting examples of disease outcomes include case loads, hospitalizations, and deaths. Examples of quantifications of distinctiveness that can be used for prediction include probability distributions of number of n-mer, divergence of such probability distributions (e.g., Cohen's D or J-S Divergence), an n-mer distinctiveness metric, a sequence distinctiveness metric, a position distinctiveness, and combinations thereof.

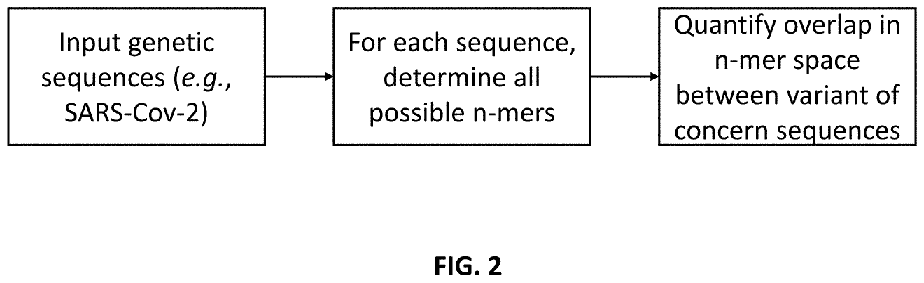

FIG. 2 shows an illustrative embodiment for analyzing distinctive n-mers in biological sequences using an alignment-free genome-based method. As shown in FIG. 2, a plurality of genetic sequences can be input, for example, genetic sequences for a virus such as SARS-Cov-2. Then, for each sequence, all possible n-mers can be determined. In some embodiments, n-mers can be identified using a sliding window with length n. Then for different sets of sequences, e.g., different variants of concern, the overlap in n-mer space can be quantified by comparing combinations of sequences from different variants. For example, a Venn Diagram can be used to show numbers of distinctive and overlapping n-mers for different combinations of variants. For example, for a given sequence from a first variant, the number of distinctive n-mers relative to one or more comparative sequences from different variants can be determined. The number of distinctive n-mers can be used, for example, to generate a probability distribution for each variant.

The methods and systems described in FIG. 2 are based on an observation that the emergence of more transmissible or immune evasive viral variants over time is associated with increased genomic distinctiveness from the strains that were previously circulating. FIG. 2 describes an illustrative method of analyzing of distinctive nucleotide n-mers in SARS-CoV-2 variants as a potential alignment-free quantification metric of viral evolution. This metric of distinctive n-mers can be used to examine SARS-CoV-2 sequence diversity, both for current variants of concern and holistically across all variants reported in the Global Initiative on Sharing Avian Influenza Data (GISAID) database. Variants that have emerged later (e.g., Delta and Omicron) indeed tend to harbor more distinct nucleotide n-mers than those that emerged earlier (e.g., Alpha, Beta, and Gamma). Although correlated, this trend is not attributable solely to overall mutational load, nor is it explained simply by a reduction in genomic distinctiveness of early VOCs due to the emergence of later VOCs. Instead, it appears that more distinctive SARS-CoV-2 variants are evolving with time, and this distinctiveness metric was investigated for its ability to predict the likelihood of newly emerging variants to out-compete those that are contemporaneously circulating in the same geographic regions. By comparing the distinctiveness of contemporary new SARS-CoV-2 lineages, the predictive potential of the distinctiveness metric is also assessed for the quick identification of emerging variants of concern.

To quantify genomic distinctiveness in a viral genome, e.g., the SARS-CoV-2 genome, including VOCs with respect to the original Wuhan strain, the number of distinctive linear nucleotide n-mers for known variants of concern can be compared. Variants of concern include Alpha, Beta, Gamma, Delta, and Omicron. Here, distinctive n-mers are n-mer sequences that occur within a specific viral lineage or set of viral genomes and that are not present in the other lineages or sets of viral genomes being evaluated, where n is the number of nucleotides in the n-mer sequence. In some embodiments, the length of n-mers is 3, 6, 9, 12, 15, 18, 21, 24, 30, 45, 60, 75, 120, 240, or in any range bounded by any value disclosed herein. In some embodiments, the length of n-mers is 9. In some embodiments, the length of n-mers is 9-30, 9-24, 18-24, or 18-30.

In some embodiments, to compare the number of distinctive n-mers for variants of concern, a plurality of combinations of sequences can be sampled, where each combination includes a sequence from each variant being compared. For each combination, all n-mers can be determined for each sequence and compared with the n-mers for the other sequences of the combination to identify distinctive n-mers not present among the other sequences of the combination. In this way, the overlap in n-space and the number of distinctive n-mers for each sequence can be determined. In some embodiments, a distribution of the probability density for the number of distinctive n-mers each variant can be used to compare distinctiveness of different variants. In some embodiments, a Venn Diagram can be used to show numbers of distinctive and common n-mers for different combinations of variants.

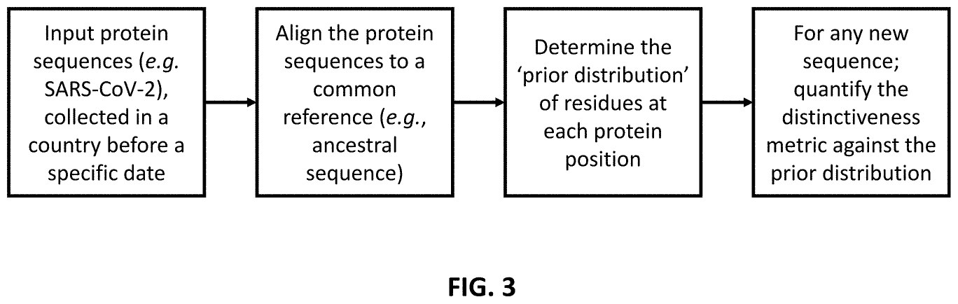

FIG. 3 shows an illustrative embodiment for analyzing distinctive n-mers in biological sequences using an aligned protein-based method. As shown in FIG. 3, a plurality of protein sequences collected from a geographic location (e.g., a country) before a given date can be input. In some embodiments, the protein sequences are from a virus such as SARS-Cov-2. Then, each protein sequence can be aligned to a common reference, e.g., an ancestral sequence. For all input sequences collected before the specific date (the “prior distribution”), the amino acid residue at each position can be determined. For any new sequence, a sequence Distinctiveness can be quantified by comparing the new sequences to the prior distribution and identifying distinctive amino acids (1-mers). For example, for a new sequence, s, its sequence Distinctiveness, D(s), is calculated using the following formula:

$$D ( s ) = \frac{1}{{N}_{p}} {\sum }_{{s}^{′} ∈ prior sequences} {\sum }_{p = 1}^{\# AA} 1 - δ ( s ( p ) - {s}^{′} ( p ) )$$

Where Nₚ is the number of prior sequences in the prior distribution, s′ is one specific prior sequence from the prior distribution, the inner sum is over all pairwise aligned amino acid positions, and δ(s(p)−s′(p)) evaluates to 1 if sequence s and s′ have the same amino-acid identity (one of twenty amino acids, a deletion, or a specific insertion) at position p and 0 otherwise.

The methods and systems described in FIG. 3 can be used to calculate sequence Distinctiveness for SARS-CoV-2 sequences based on a proteome-wide comparison with all prior sequences from the same geographical region. In some embodiments, sequence Distinctiveness relative to contemporary sequences is correlated future change in prevalence of that sequence's lineage, suggesting that the Distinctiveness of emergent SARS-CoV-2 lineages is associated with their epidemiological fitness. In some embodiments, the sequence Distinctiveness metric can give an intuitive understanding of the immunological distinctiveness of a given variant and relative Distinctiveness is strongly correlated to competitive fitness. In some embodiments, the use of sequence Distinctiveness in real-time assessment of viral genomes can be used for pandemic preparedness initiatives. In some embodiments, sequence Distinctiveness can capture both the emergence of new amino-acid substitutions (e.g., D614G) and deletion of sequence regions that may be involved in antibody recognition, both of which can affect viral sequence immunogenicity and infectivity. In some embodiments, sequence Distinctiveness can predict future changes in local prevalence of newly circulating lineages, suggesting that sequence Distinctiveness could contribute to accurate and early identification of newly circulating lineages that are likely to outcompete other contemporary lineages. In some embodiments, the methods described in FIG. 3 can include a correction for the durability of immunity, for example, by reduced contributions to the sequence Distinctiveness calculation of sequences based on their collection date. For example, the contribution of more recent sequences can be reduced. In one example, the following weight can be applied to a sequence based on collection date:

e^{-At}

where A is a coefficient and t is the time (e.g., days) since the collection date.

In some embodiments, the methods and systems described herein include a user interface (UI). A user interface can allow a user to select variants for analysis and select a sample size (which determines the number of sets or combinations of variants are analyzed). Additionally, a user can define a time period so that the system analyzes sequences collected during that time period. In this way, the user can compare variants that were circulating at the same time or analyze whether the distinctiveness of variants change over time. Alternatively, the user can segment sequences based on any other metadata associated with the sequence data, including geography or sublineages. For example, by analyzing sequences from different geographical regions, the user can compare distinctiveness of variants circulating in different regions. Alternatively segmenting variants in this way may indicate distinct groups within variants.

In some embodiments, a user interface allows a user to visualize results of distinctive n-mer analysis. For example, the user interface generates a distribution showing the probability density function for the number of distinctive n-mers for each variant selected. In addition, the user interface generates a Venn diagram for the selective variants to show the number of distinctive and overlapping n-mers.

### Examples

Certain embodiments will now be described in the following non-limiting examples.

### Alignment-Free, Genome Based Analysis of SARS-Cov-2 Variants

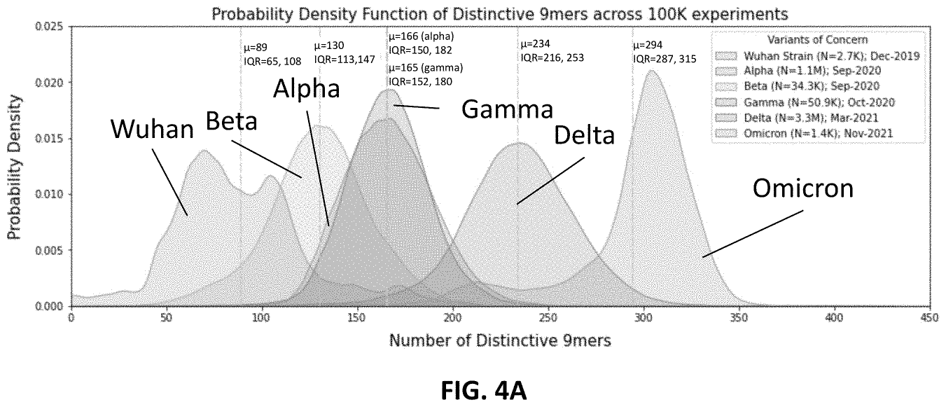

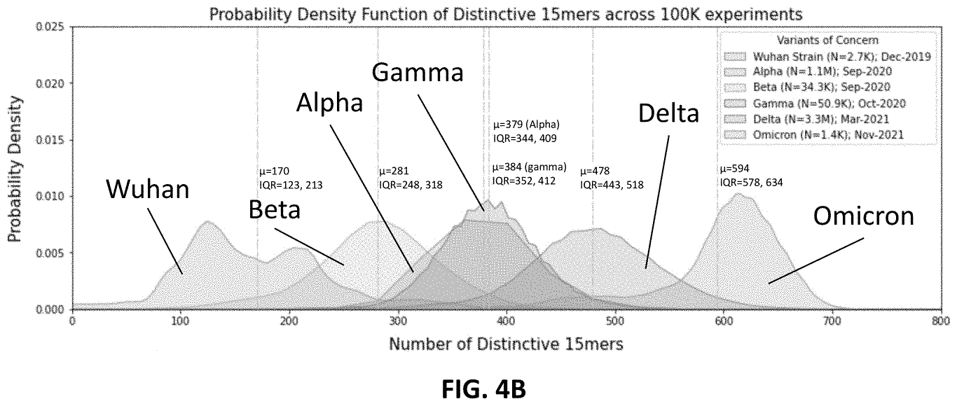

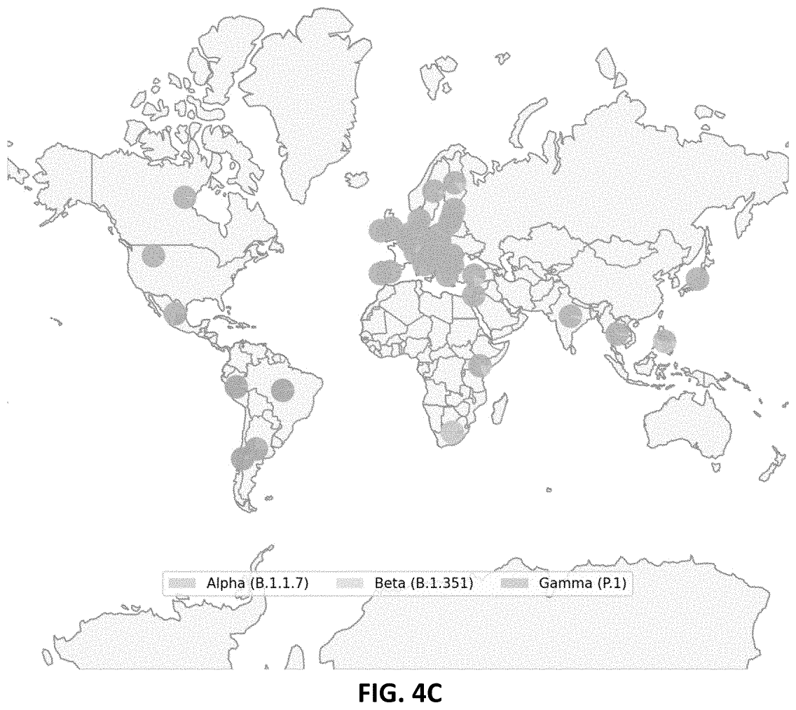

In the examples shown in FIGS. 4A-17F, distinctive nucleotide n-mers in SARS-CoV-2 variants were analyzed as a potential alignment-free quantification metric of viral evolution. This metric of distinctive n-mers can be used to examine SARS-CoV-2 sequence diversity, both for current variants of concern and holistically across all variants reported in the Global Initiative on Sharing Avian Influenza Data (GISAID) database.

To quantify distinctive n-mers, a polynucleotide sequence analysis was performed for SARS-CoV2 variants of concern. Sequences from 6 variants of concern (Wuhan reference, Alpha, Beta, Gamma, Delta, and Omicron) were calculated for sequences obtained from the GISAID database. For this analysis, repetitions of an iterative sampling experiment were performed in which one genome assigned to each lineage was selected for each iteration, a set of n-mers from each genome was derived, and then the genomes in these sets were compared against each other to determine the number of distinctive nucleotide n-mers. In one example, sequences were sampled with replacement for each variant of concern to generate 100,000 sets of 6 sequences. In other embodiments where the number of sequences available is greater, sampling can be done without replacement. For variants where the number of sequences available is limited, sampling with replacement can lead to oversampling for these variants. The overlap of 9-mer sequences is calculated for each of the 100,000 sets of 6 sequences to generate a distribution of distinctive n-mersequences. This procedure was repeated for n-mer sequences of various lengths.

The following method was used for polynucleotide analysis
1. 6 variants of concern were considered: Wuhan reference, Alpha, Beta, Gamma, Delta and Omicron. The Pango lineages for these variants are expanded using this link (https://www.cdc.gov/coronavirus/2019-ncov/variants/variant-classifications.html).
2. From GISAID, 100K sequences were sampled from each of these 6 variants. Sampling with replacement is done for the Wuhan Strain, Beta, Gamma, and Omicron.
3. This allows creation of 100K sets or combinations of 5 different variant sequences. For each set, the overlap in the number of 9-mers is computed, and distinctive n-mers for each variant are identified. A 5-way Venn diagram is constructed for each set of 5 sequences that identifies the number of n-mers in each region of the Venn diagram. Analysis of these 100 k set is analogous to performing 100K “experiments.”
4. Doing this across all 100K sets gives a distribution for each region in the Venn diagram and gives a distribution for the distinctive n-mers for each variant.
5. In this example, the distribution of distinctive 9-mers increases from the Wuhan reference to Omicron. This recapitulates earlier preliminary analysis. Here, note that the ‘N=’ numbers refer to the number of unique sequences available for each variant.
6. The above steps were repeated for 3-mers and 15-mers. In the case of 3-mers, there were no distinctive n-mers for any variant. The 15-mer distribution was also plotted.

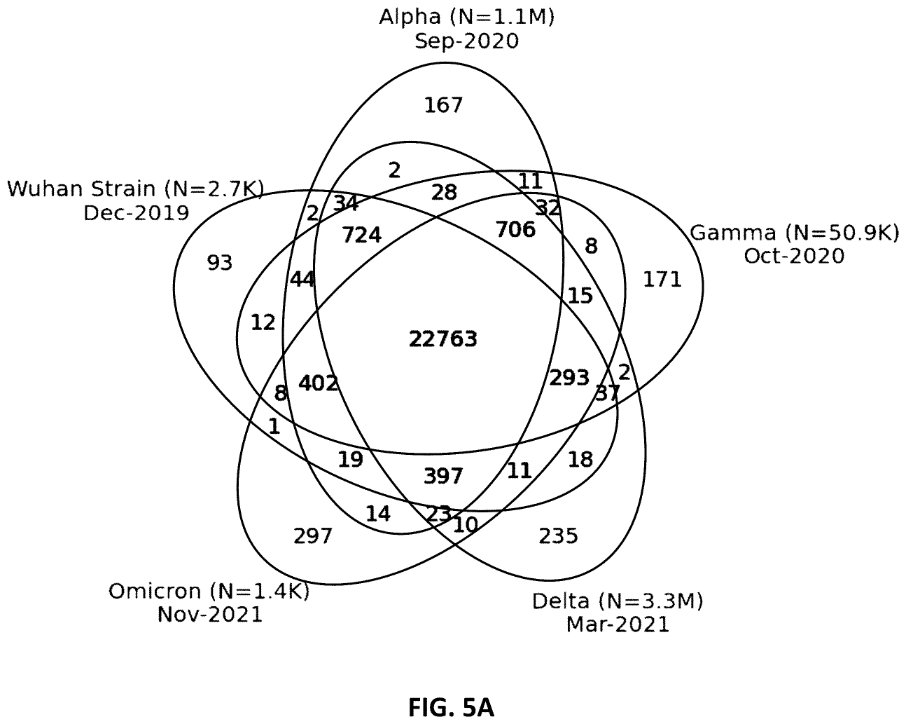

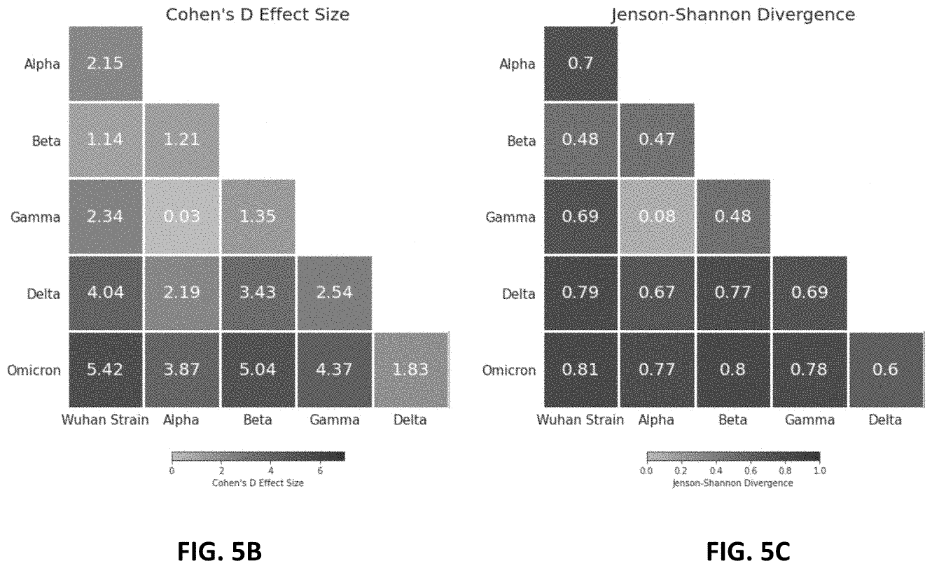

FIGS. 4A-4B show the distribution of the number of distinctive n-mers obtained for each lineage when randomly sampling representative sequences for each lineage from the full GISAID database (100,000 samples of 6 sequences). An increase in the frequency of distinctive n-mers was observed for more recent variations of concern (e.g., Delta and Omicron). As shown in FIG. 5A, distinctive n-mers are indicated in the region of the Venn Diagram for each variant that does not overlap with any of the other variants plotted.

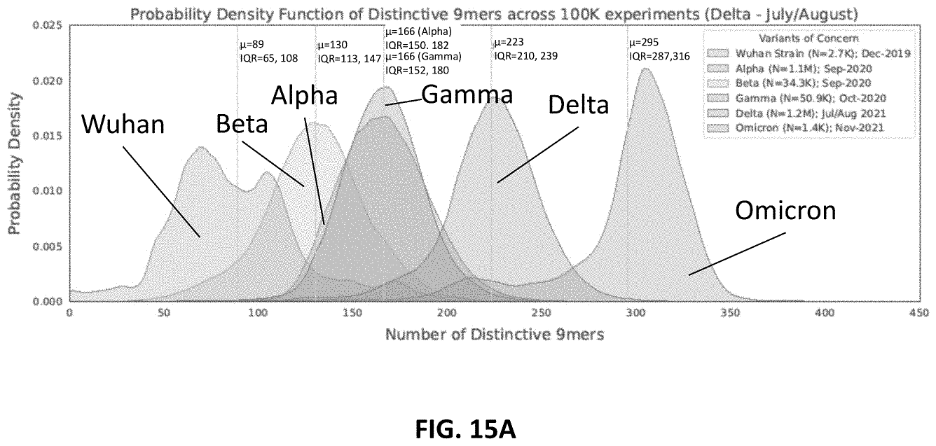

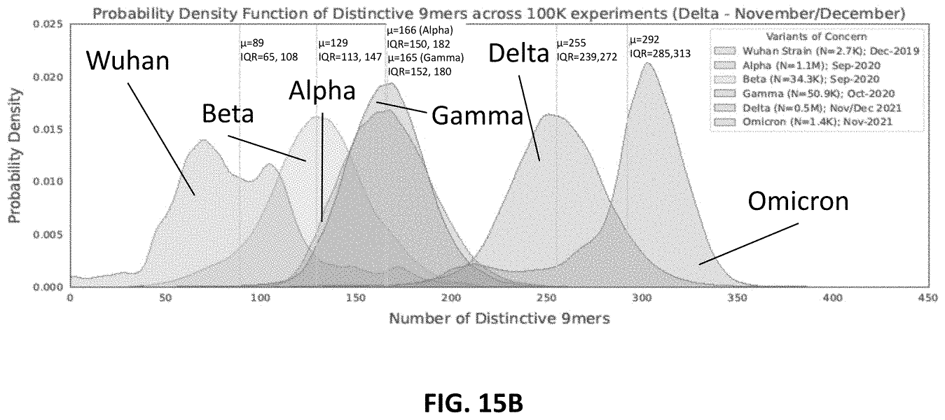

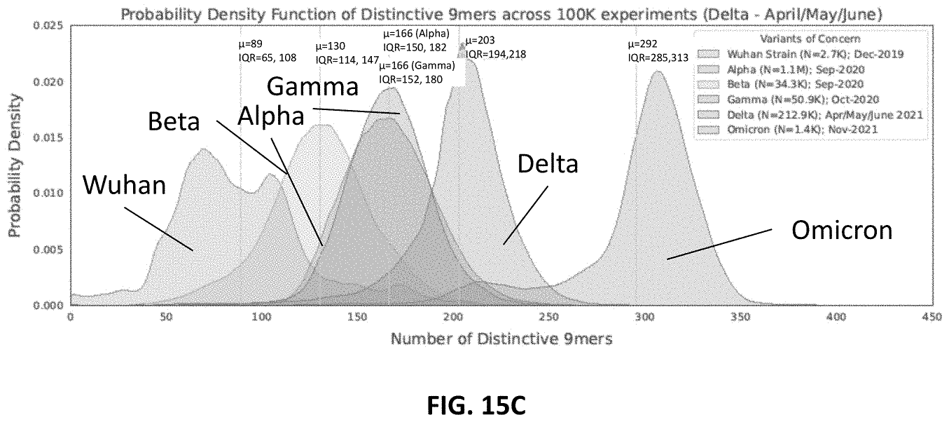

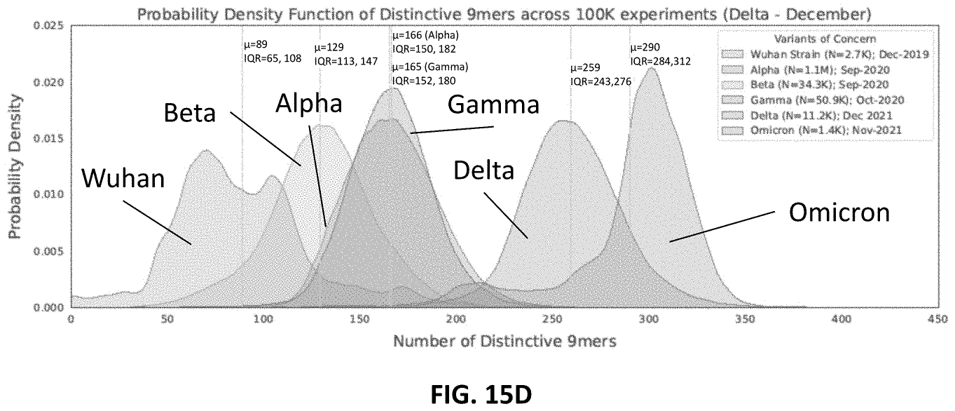

FIGS. 4A-4B show distribution of 9-mer (FIG. 4A) and 15-mer (FIG. 4B) sequence diversity. Density plots show 9-mer and 15-mer sequence diversity, as measured by the number of distinctive polynucleotide sequences in SARS-CoV2 variants of concern. In FIGS. 4A-4B, the distributions for each variant are reasonably well-separated, suggesting that distinctive 9-mers and 15-mers are each able to identify emerging variants. However, the separation for the distinctive 9-mers, shown in FIG. 4A, is greater than the separation for the distinctive 15-mers, shown in FIG. 4B. In both FIG. 4A and FIG. 4B, the Wuhan distribution is bimodal, suggesting that the viral genomes that were classified as “Wuhan reference” could be split into at least two groups. This indicates that, in some embodiments, distinctive n-mers could be used to identify variants.

FIGS. 4A-4B show an increase in the number of distinctive n-mers shown for variants of concern that emerged later; this trend is observed for 9-mers (FIG. 4A), 15-mers (FIG. 4B) and all other n-mer sizes tested. As shown in FIG. 4A, distinctive 9-mers for each of the plotted variants correlates with the date at which these variants first emerged. For example, the Omicron variant (emerged November 2021) has the highest number of distinctive 9-mers, followed in order by the Delta (March w2021), Gamma (September 2021), Alpha (October 2021), and Beta variant (September 2021) with the Wuhan reference showing the least distinctive 9-mers. Thus, the number of distinctive 9-mers increased over time as new variants emerged, indicating that the variants are diverging from the Wuhan reference. The apparent correlation between time of emergence and the number of distinctive 9-mers for a variant is likely the result of their relative positions in the viral phylogenetic tree. Lineages derived from a common ancestor will all overlap with parts of this ancestor, thereby reducing the distinctiveness of the ancestor.

FIG. 4C shows geographic distribution of Alpha, Beta, and Gamma variants based on sequences deposited in GISAID through 14 Dec. 2021. In FIG. 4C, each circle represents the proportion of Alpha, Beta or Gamma sequences deposited in the country. Only countries where at least 1000 sequences are deposited are shown. FIG. 4C shows that Gamma and Beta variants, and to a large extent the Alpha variant, circulated in generally distinct regions around the same time periods. Alpha, Beta, and Gamma emerged at similar times (September-October 2021) and drove surges in largely distinct regions (Alpha in the United States, United Kingdom, and Asia; Beta in South Africa; Gamma in South America). On the other hand, after Delta emerged and drove one of the largest national surges to date in India during April-May 2021, it became the dominant strain across the globe for the next several months. Similarly, Omicron rapidly spread across the globe shortly after its emergence in November 2021 and has already become the dominant variant in most regions.

FIGS. 5A-5C show additional ways to visualize distinctive 9-mers from the distributions in FIG. 4A. FIG. 5A shows a Venn diagram showing the number of distinctive 9-mer sequences for SARS-CoV2 variants of concern. The Venn diagram shows the mean of the distribution (across 100K experiments) for each combination of variants. Note that beta is not included in the Venn diagram because Venn diagrams with more than 5 variants do not provide clear visualization. The center of the Venn diagram shows the number of 9-mers that are common among the five variants (22,763), indicating that there remains a lot of overlap between the SARS-COV-2 variants. In some embodiments, monitoring n-mers that are at the center of the Venn Diagram can be used to identify portions of the viral genome that are stable or that are not changing as variants emerge. The outer edges of the Venn diagram show the number of distinctive n-mers for each variant which are not present in the other variants.

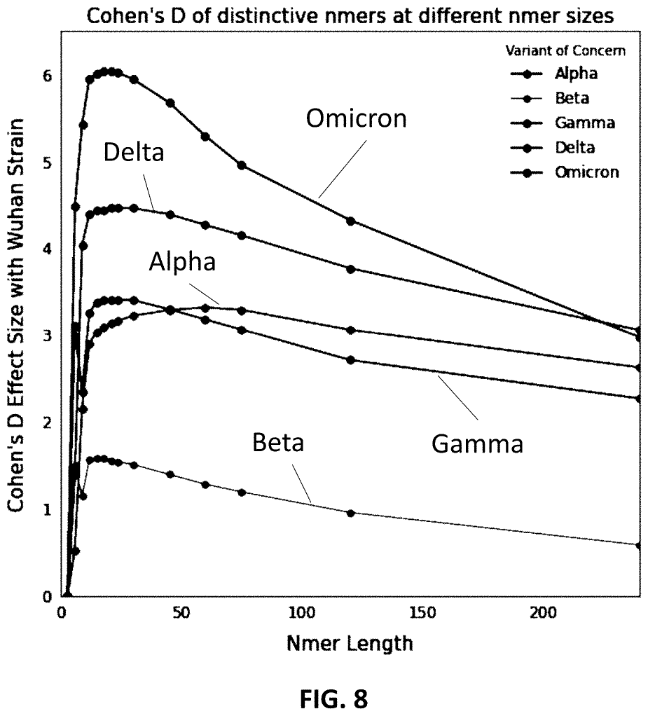

Based on the Cohen's D (effect size) and Jensen-Shannon divergence (total divergence to the average) values, shown in FIGS. 5B-5C, the distinctive 9-mer metric was found to distinguish between variants of concern, and variants of interest, discussed below in relation to FIG. 8. FIGS. 5B-5C show heatmaps of Cohen's D (FIG. 5B) and Jensen-Shannon (J-S) divergence (FIG. 5C) values from pairwise comparisons of the distribution of distinctive 9-mer sequences between SARS-CoV2 variants of concern. The Cohen's D and J-S Divergence were computed for each pair of distributions of distinctive n-mers plotted in FIG. 4A. For Cohen's D, the absolute value is shown. In this example, there are significant non-zero Cohen's D values and Jensen-Shannon divergence values between all pairs of variants of concern, other than Alpha and Gamma, which emerged near the same time in SARS-CoV-2 evolution, during September 2020 and October 2020 respectively. Consistent with the data shown in FIGS. 4A-4B, the Omicron variant shows the greatest divergence from the reference Wuhan strain, with Cohen's D=5.42 and J-S divergence=0.81. Omicron sequences robustly had more distinctive 9-mers than all other VOCs (Cohen's D>3 for each comparison), and Delta sequences had more distinctive 9-mers than all VOCs other than Omicron. However, as shown in FIG. 5B, Alpha, Beta, and Gamma showed more overlap, with all Cohen's D values less than 1.5 relative to each other. A similar pattern was also observed when comparing the distinctive 9-mer distributions via Jensen-Shannon divergence, as shown in FIG. 5C.

Other quantities for distinguishing between SARS-CoV-2 variants were also evaluated for their ability to distinguish between the variants of concern in comparison to distinctive n-mers. These metrics included mutational load and phylogenetic distance. None of the other metrics tested had the resolution to discriminate between all pairs of variants of concern.

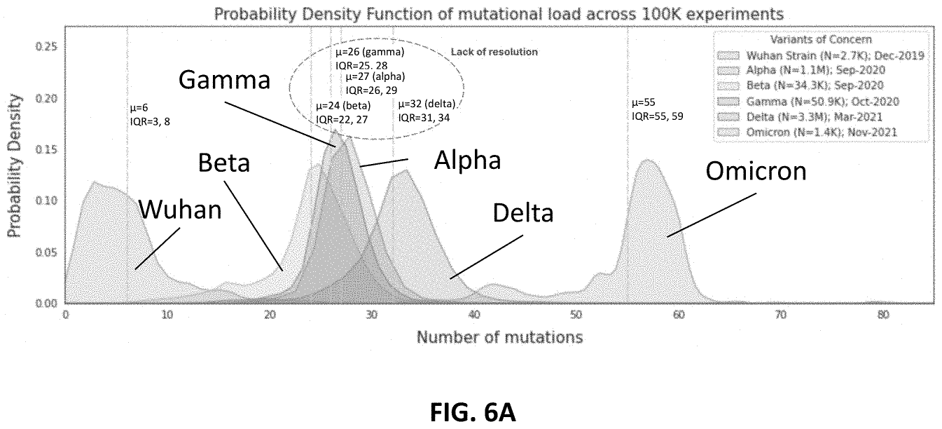

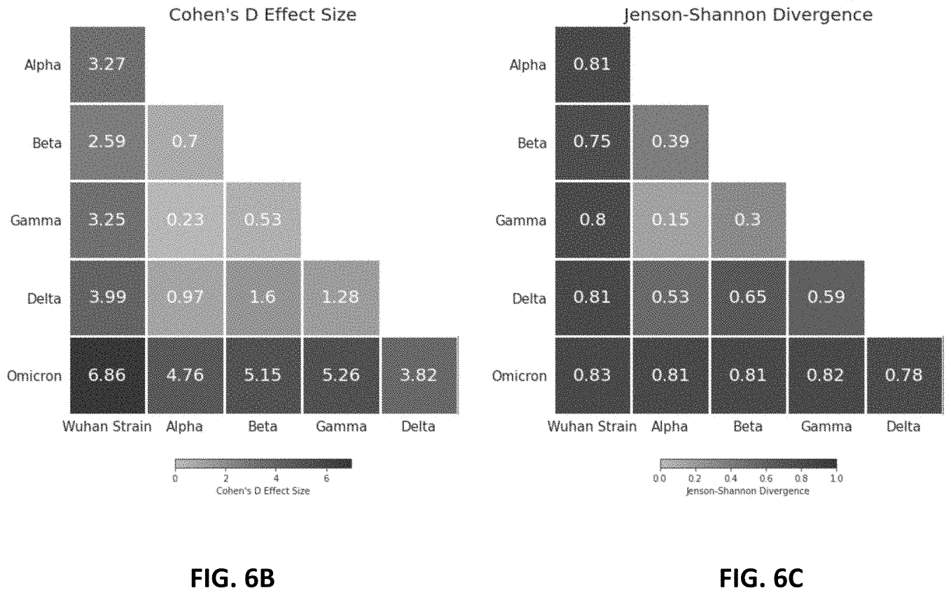

As shown in FIGS. 6A-6C, the distribution of mutational load within variants of concern was further examined. For a given sequence, the mutational load is the number of amino-acid level mutations in a sequence relative to a reference sequence. For sequences obtained from the GISAID database, the number of novel mutations relative to the original Wuhan variant of SARS-CoV2 were examined. For each variant of interest, a distribution of the number of novel mutations was generated and pairwise comparisons were performed between variants by calculating Cohen's D and Jensen-Shannon divergence. For each sequence in the random sample, the number of mutations for each variant of concern was obtained from GISAID. Mutations across the entire transcriptome were considered.

The distributions of mutational load for each variant of concern were plotted in FIG. 6A, and the Cohen's D and J-S divergence between these distributions are shown in the heatmaps in FIGS. 6B-6C. The Omicron variant had the highest frequency of mutations among variants of concern (FIG. 6A). Moreover, the distribution of the number of mutations show that the Omicron variant has the largest sequence divergence from the Wuhan Strain (FIGS. 6B-6C). However, as shown in FIG. 6A, the mutational load fails to distinguish or provide resolution between the Alpha, Beta, and Gamma variant. The mutational load shows only a small difference between those three variants and the Delta variant, with mean values of 27, 26, 24, and 32, respectively. This is also reflected in FIGS. 6B-6C, which shows the Cohen's D values and J-S divergence values calculated for mutational load. The mutational load is highly correlated with the distinctive n-mer metric; however, as shown in FIGS. 4A-4B and 5B-5C, there was greater resolution between these variants based on the distinctive n-mer metric than between variants based on the mutational load. This indicates that the number of distinctive n-mers harbored by a VOC provides additional information about emerging variants, beyond what is available from mutational load or the number of mutations away from the reference SARS-CoV-2 genome.

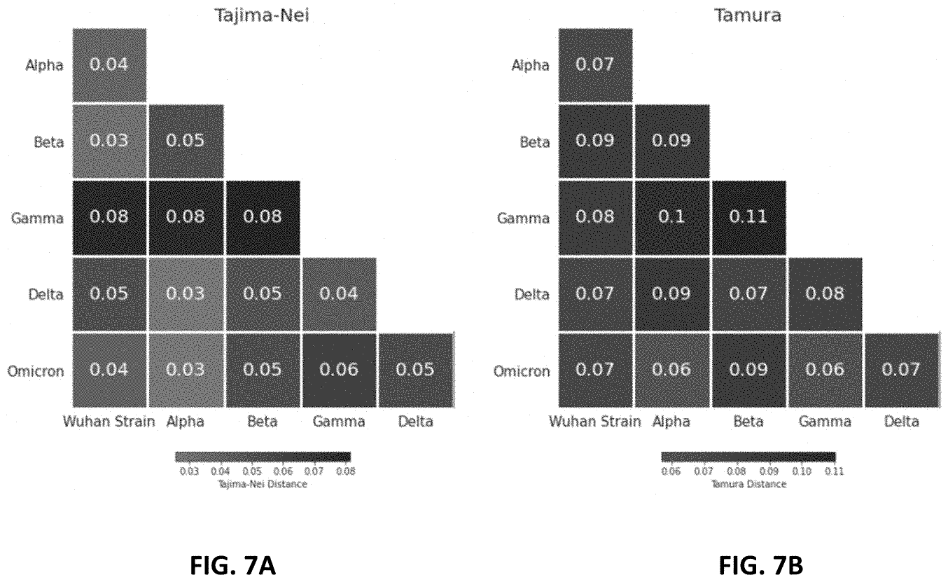

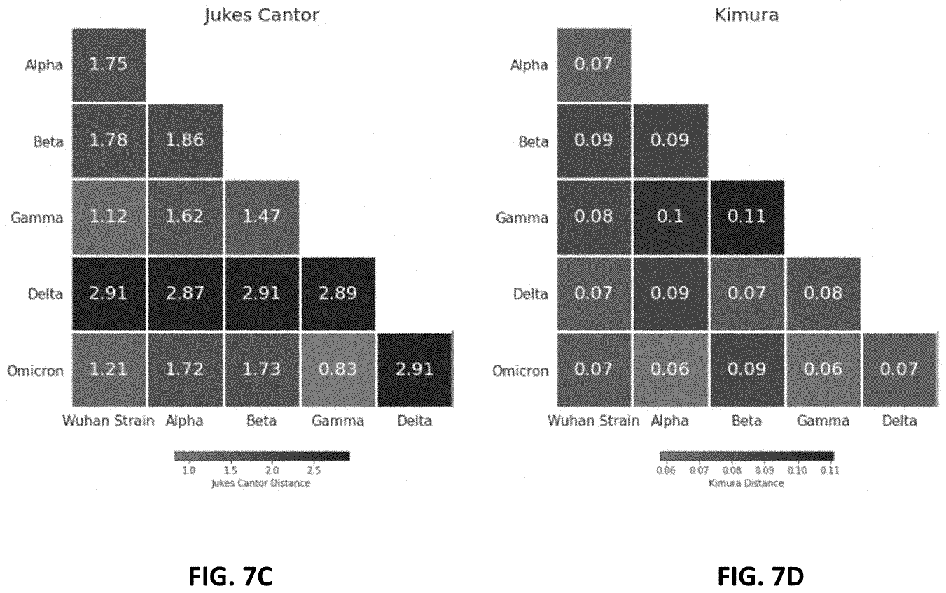

As shown in FIGS. 7A-7D, the Tajima-Nei, Tamura, Jukes-Cantor, and Kimura metrics, commonly used in phylogenetic analysis, were tested, and none of these metrics provided resolution between all tested variants of concern. As is known in the art, each of these metrics estimates the average number of nucleotide substitutions per site between homologous sequences, with the Jukes-Cantor method assuming equal base frequencies and equal mutation rates, the Kimura metric distinguishing between transitions (from purine to purine or pyrimidine to pyrimidine) and transversions (from purine to pyrimidine or pyrimidine to purine), the Tajima-Nei metric taking into account unequal rates of substitution among different nucleotide pairs, and the Tamura metric extending the Kimura metric to a case where G+C bias exists. In each approach, as shown in FIGS. 7A-7D, the average phylogenetic distance between variants of concern was calculated. Phylogenetic trees were constructed to understand the relationship between strains and observe the Omicron strain to be a distinct lineage. Phylogenetic trees were constructed using a distance matrix generated using FASTA sequences, shown in Table 1 below. From the aligned FASTA sequences available in nextstrain.org, run the following methods:
1. Number of sequences are shown in Table 1.
2. From the aligned FASTA sequences available in nextstrain.org, run the tree construction code and generate the distance matrix.
3. For each pair of lineages (15 combinations), obtain the mean phylogenetic distance of all pairs of sequences. This mean distance is shown in the heatmaps in FIGS. 7A-7D.
4. Measure phylogenetic distance using four different measures/models: Tajima-Nei, Tamura, Jukes Cantor, Kimura.

TABLE 1
Number of sequences used to generated phylogenetic Trees

| Variant | Number of Sequences |
|---|---|
| Wuhan Strain | 12 |
| Alpha | 97 |
| Beta | 20 |
| Gamma | 28 |
| Delta | 1380 |
| Omicron | 2 |

FIGS. 7A-7D show analysis of existing phylogenetic distance measures. These existing measures of phylogenic distance show lack of resolution in identifying similar/distinct lineages. This indicates that quantifying distinctive n-mers provides additional information about emerging variants, beyond what is available from phylogenetic distance measures. FIG. 7A-7D show phylogenetic reconstruction of SARS-CoV2 Variants of Concern using the following methods: Tajima-Nei, Tamura, Jukes-Cantor, and Kimura. The heatmaps show the mean phylogenetic distance calculated between SARS-CoV2 variants of concern using the Tajima-Nei (FIG. 7A), Tamura (FIG. 7B), Jukes-Cantor (FIG. 7C), and Kimura (FIG. 7D) methods. In 3 out of 4 distance measures, Alpha-Gamma phylogenetic distance is higher than Alpha-Beta distance. This is opposite of what is observed using distinctive n-mers. For each comparison, the Tamura and Kimura metrics return very similar values to each other.

FIG. 8 shows the relationship between Cohen's D and n-mer lengths. Cohen's D values of the distinctive 9-mer distributions of the Alpha, Beta, Gamma, Delta, Omicron variants were plotted against the Wuhan Strain for different n-mer lengths. The distributions of distinctive n-mers were computed for n-mers of size 3, 6, 9, 12, 15, 18, 21, 24, 30, 45, 60, 75, 120, 240. FIG. 8 shows the absolute value of Cohen's D for the distinctive n-mer distribution of the Wuhan strain compared with those of the Alpha, Beta, Gamma, Delta and Omicron strains. Cohen's D increases and then peaks at n-mer size of about 18-24, after which it decreases. Although most examples presented herein use 9-mer polynucleotides, FIG. 8 shows that different n-mer size would yield similar conclusions for polynucleotide sequences, and the results based on 9-mers are therefore illustrative. The highest Cohen's D value for each variant is marked with an asterisk in Table 2. In some embodiments, the optimal length of an n-mer is determined by analyzing the Cohen's D or the J-S divergence as a function of n-mer length.

TABLE 2
Cohen's D values as a function of n-mer length for variants
of concern (highest value indicated with an asterisk)

| N-mer length | Alpha | Beta | Gamma | Delta | Omicron |
|---|---|---|---|---|---|
| 3 | 0 | 0 | 0 | 0 | 0 |
| 6 | 0.53 | 1.5 | 3.11 | 1.4 | 4.49 |
| 9 | 2.15 | 1.14 | 2.34 | 4.04 | 5.42 |
| 12 | 2.9 | 1.56 | 3.26 | 4.4 | 5.95 |
| 15 | 3.04 | 1.58* | 3.38 | 4.43 | 6 |
| 18 | 3.09 | 1.58* | 3.41* | 4.43 | 6.03* |
| 21 | 3.13 | 1.56 | 3.4 | 4.46* | 6.03* |
| 24 | 3.17 | 1.54 | 3.4 | 4.46* | 6.01 |
| 30 | 3.22 | 1.51 | 3.4 | 4.46* | 5.95 |
| 45 | 3.29 | 1.4 | 3.3 | 4.39 | 5.68 |
| 60 | 3.32* | 1.28 | 3.18 | 4.27 | 5.29 |
| 75 | 3.29 | 1.19 | 3.06 | 4.15 | 4.95 |
| 120 | 3.06 | 0.96 | 2.71 | 3.77 | 4.32 |
| 240 | 2.63 | 0.58 | 2.27 | 3.06 | 2.97 |

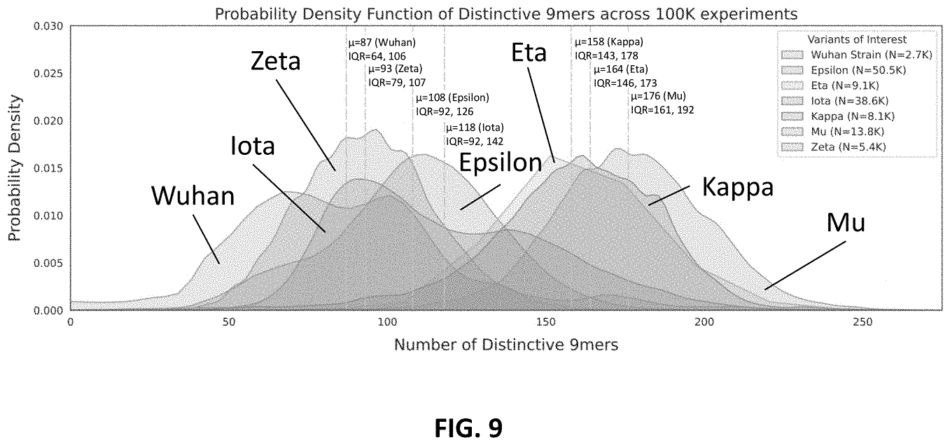

FIG. 9 shows distinctive 9-mers for Variants of Interest (VOIs). The number of distinctive 9-mers is higher for recent variants of concern than the number of distinctive 9-mers for variants of interest, which have a mean number of distinctive 9-mers of 93 for Zeta, 103 for Epsilon, 118 for Iota, 158 for Kappa, 164 for Eta, and 176 for Mu. These variants were analyzed same procedure as described for the variants of concern. These procedures were applied to the variants of interest: Epsilon, Eta, Iota, Kappa, Mu, Zeta and the Wuhan Strain. Lineages were expanded using https://www.cdc.gov/coronavirus/2019-ncov/variants/variant-classifications.html. As shown in FIG. 9, there are two groups. Group 1 includes Wuhan, Epsilon, Zeta, Iota, while group 2 includes Kappa, Mu and Eta. The variants of Group 1 overlap with the Wuhan reference. Since VOIs have lower numbers of distinctive 9-mers compared to VOCs, the distinctive 9-mer metric can be used to identify new emerging VOCs, e.g., by identifying a variant initially classified as a VOI that has a higher number of distinctive n-mers than other VOIs.

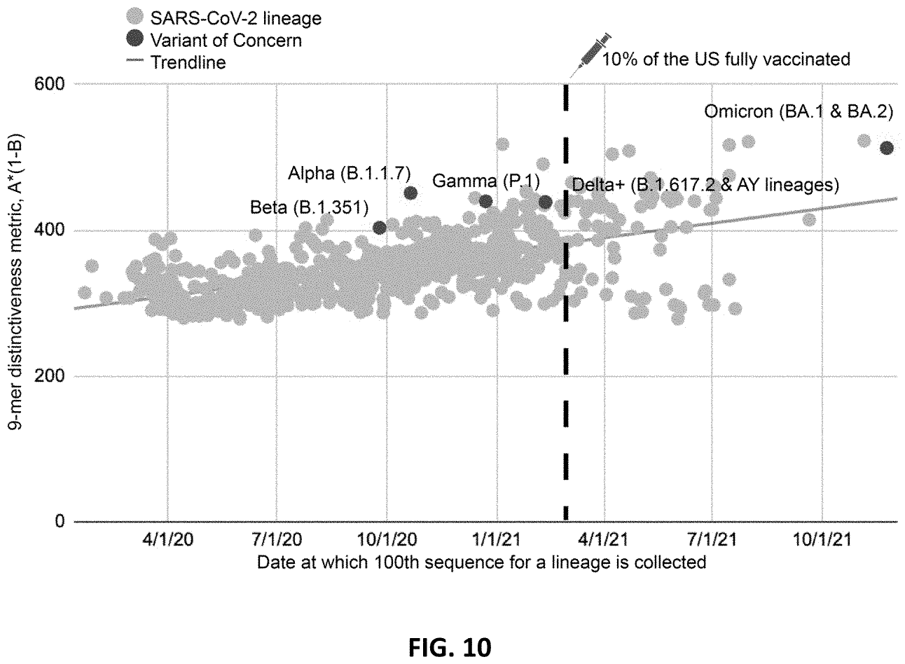

FIG. 10 shows temporal trends in polynucleotide sequence diversity. The relationship between the number of distinctive 9-mers for SARS-CoV-2 variants and how far evolved a variant is compared to the root Wuhan-Hu-1 variant was analyzed. A metric, A*(1−B), related to the distinctive 9-mer metric, was evaluated for all ˜3.5 million complete SARS-CoV-2 transcriptome sequences reported in GISAID. For a given lineage, 1, the following n-mer distinctiveness metric was calculated:

l=<A(l,n)*(1−B(l,n))>ₙ,

where A(l,n) is the fraction of sequences in GISAID of lineage l that contain a specific n-mer, n, and B(l,n) is the fraction of sequences in GISAID of all lineages except lineage l that contain n-mer n. The angular brackets indicate averaging over all n-mers that are reported for the lineage. As shown in FIG. 10, this metric is correlated with the date at which a lineage first emerged, supporting that the distinctive n-mer metric is a metric of evolutionary drift from the root lineage (here, the Wuhan Lineage). FIG. 10 also included a dashed line indicating when 10% of the US was fully vaccinated. Furthermore, variants of concern (Alpha, Beta, Gamma, and Omicron) exhibit high values in this metric, compared with other contemporary lineages (dark grey dots indicating variants of concern in FIG. 10).

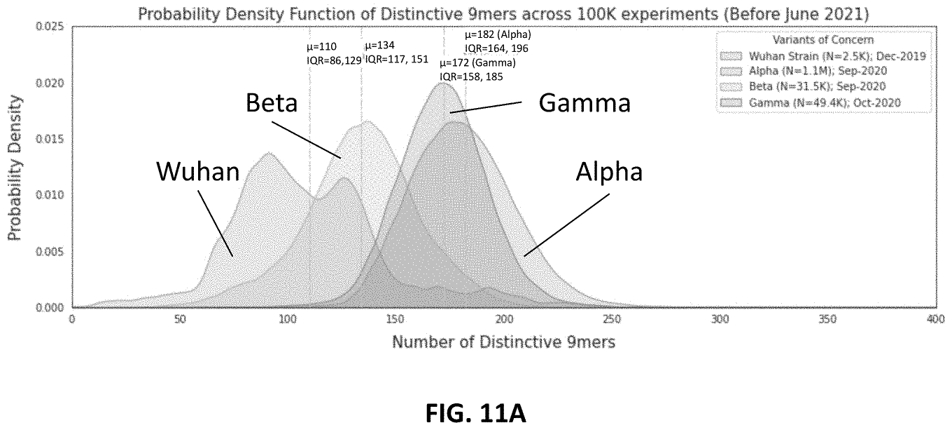

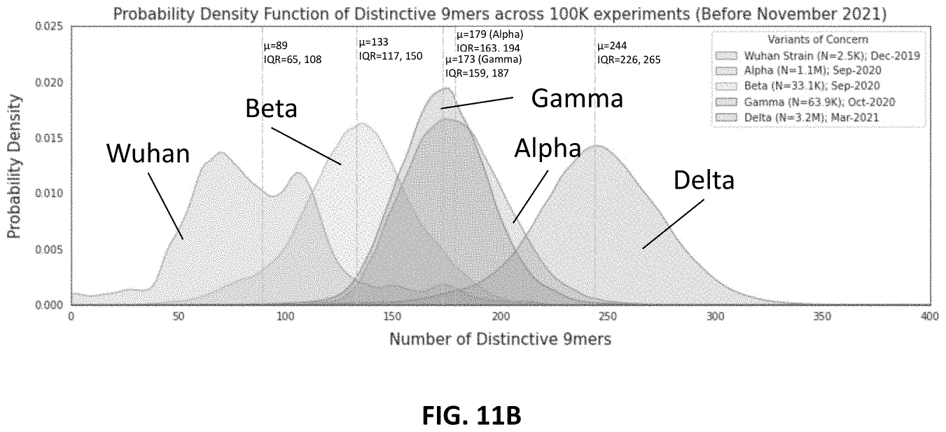

In some embodiments, distinctive n-mers can be analyzed for sequences collected during particular time windows. FIGS. 11A-11B shows the distinctiveness of variants before and after the onset of the Delta variants. Three cases were analyzed: (1) distinctiveness between Wuhan strain, Alpha, Beta and Gamma before June 2021 (i.e. before Delta started surging), (2) distinctiveness between Wuhan strain, Alpha, Beta, Gamma and Delta before November 2021 (i.e. before Omicron started surging), (3) distinctiveness between Wuhan strain and Delta variant in July-August 2021 compared to the distinctiveness between Delta variant in November-December 2021 and Omicron variant.

FIG. 11A shows the 9-mer distinctiveness of the Wuhan strain, Alpha, Beta, and Gamma variants before June 2021, before Delta emerged. FIG. 11B shows the 9-mer distinctiveness of the Wuhan strain, Alpha, Beta, Gamma, and Delta variants before November 2021, after Delta emerged. FIG. 11A and FIG. 11B are similar. When FIG. 11B is compared to FIG. 11A, the 9-mer distinctiveness of Wuhan Strain, Alpha, Beta and Gamma variants are unchanged after onset of the Delta variant.

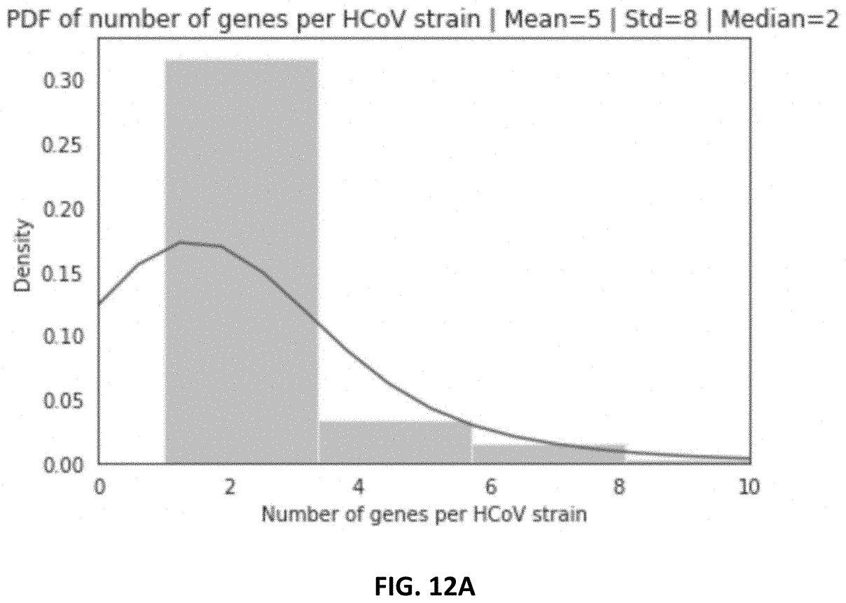

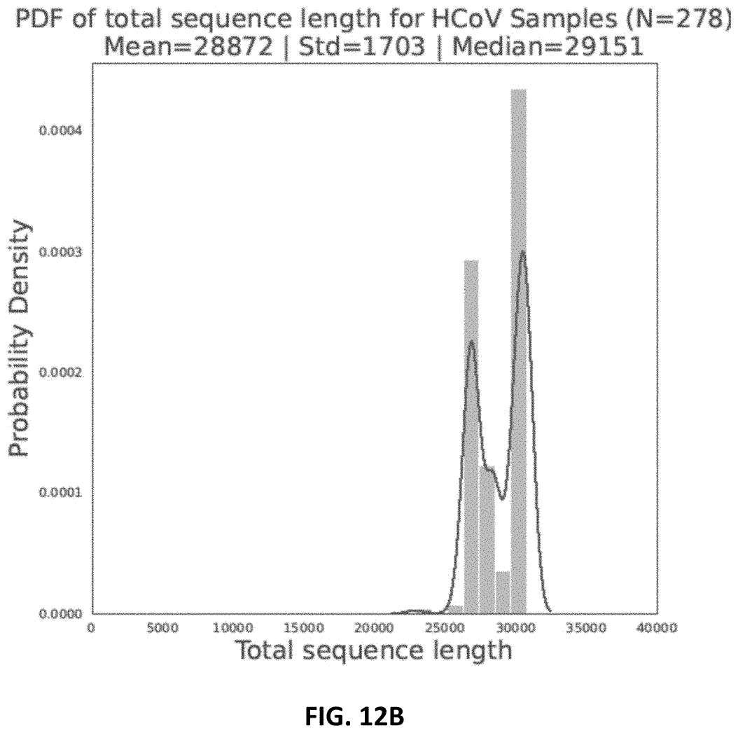

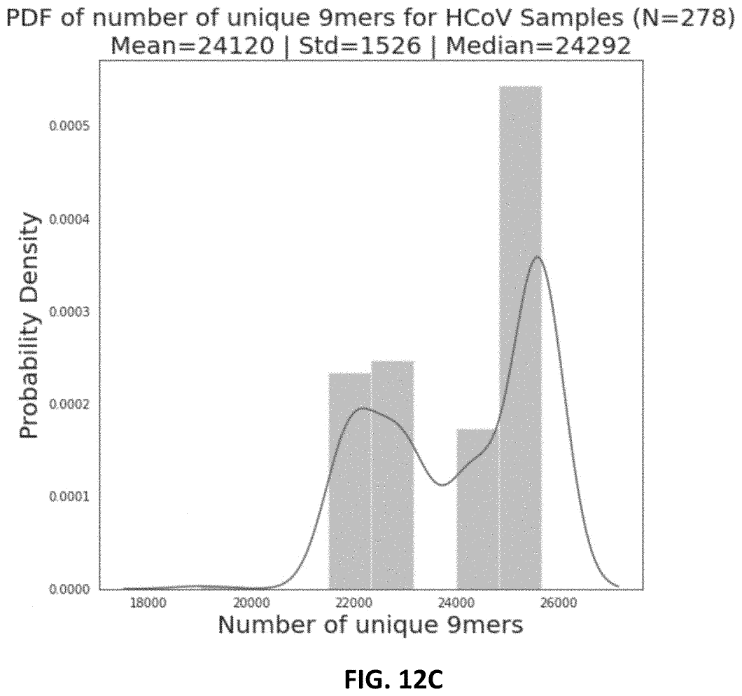

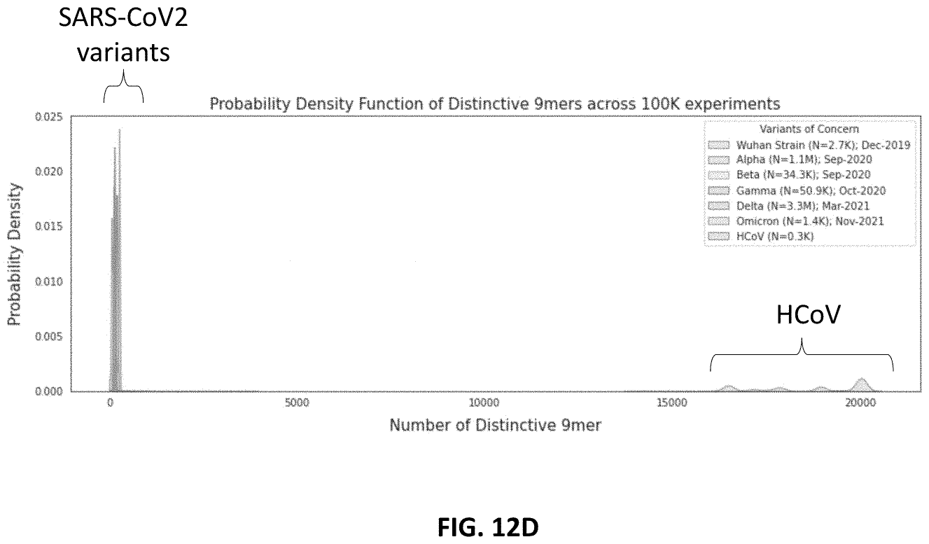

FIGS. 12A-12D shows mapping of seasonal coronaviruses (human coronaviruses—HCoVs) onto the SARS-CoV-2 genomic polynucleotide distribution. HCoV coding transcriptomes were obtained from https://www.viprc.org/. In this dataset, there were a total of 3,369 HCoV strains across four HCoV species: 229E (N=32), OC43 (N=174), NL63 (N=64), HKU1 (N=18). FIG. 12A shows a probability density function of the number of genes per HCoV strain. There was a mean of 5, a standard deviation of 8, and median of 2. FIG. 12B shows a probability function of total sequence length per HCoV strain. There was a mean of 28872, a standard deviation of 1703, and a median of 29151. FIG. 12C shows a probability density function of the number of distinctive 9-mers per HCoV strain. There was a mean of 24120, a standard deviation of 1526, and a median of 24292. FIG. 12D shows a probability function of the number of distinctive 9-mers for HCoV compared to variants of concern. FIGS. 12C-12D indicate that seasonal coronavirus variants are more distinctive than SARS-CoV-2 variants of concern. HCoV being a different species is seen to be far more distinct than the SARS-CoV-2 variants. The mean number of distinct HCoV 9-mers was 18654 with a standard deviation of 1436.

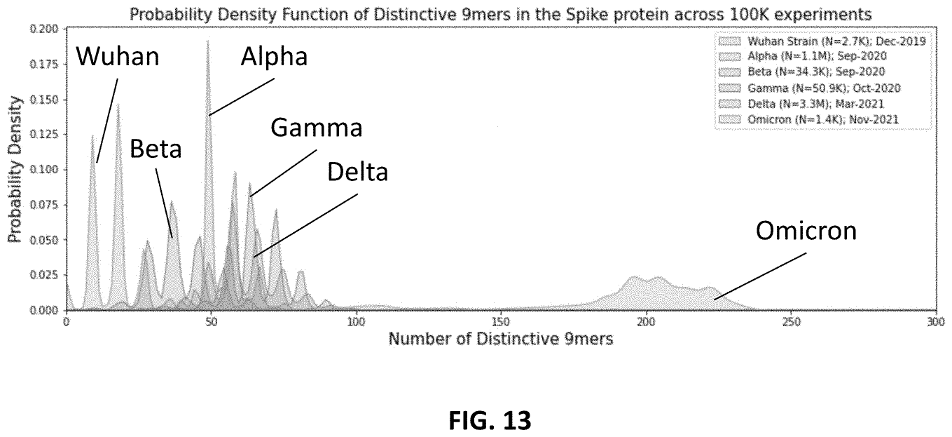

Distinctive 9-mers from the region of the viral genome encoding the spike protein were also investigated. FIG. 13 shows density plots of 9-mer sequence diversity for 9-mers in the spike protein, as measured by the number of distinctive polynucleotide sequences in SARS-CoV2 variants of interest. Gamma and Delta are very similar, and Delta in fact has slightly a lower number of distinctive 9-mers than Gamma. Omicron has a much higher number of distinctive 9-mers in the region of the spike protein than the other variants of concern. Many of the variants have multimodal distributions.

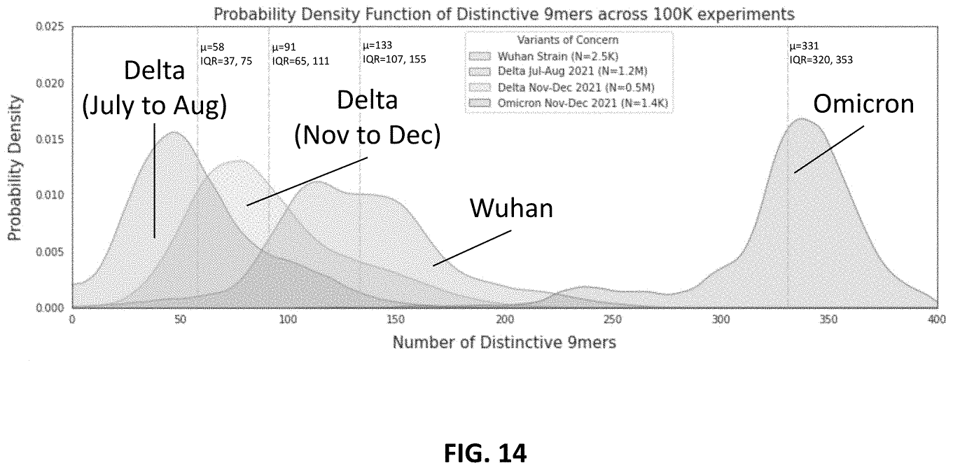

Viral lineages can be split further into subgroups based on any associated metadata. For example, as shown in FIG. 14, a variant can be split into two groups based on the time sequences were collected. FIG. 14 shows density plots of 9-mer sequence diversity for the Wuhan strain, Delta, and Omicron, with Delta split into two subgroups based on time. The first Delta subgroup includes sequences collected from July to August of 2021, while the second Delta subgroup includes sequences collected from November to December of 2021. As shown in FIG. 14, the distinctiveness of each of these subgroups is lower than the undivided Delta variant (as shown in FIG. 4A). The distributions of these subgroups are shifted left (toward the Wuhan distribution) compared to the original undivided Delta distribution in FIG. 4A. This result may indicate that the two Delta subgroups analyzed have overlapping n-mers. These results indicate that the distinctive n-mer metric can pick up when highly distinct lineages are present.

FIGS. 15A-15D show the probability density functions of distinctive 9-mers for Wuhan, Alpha, Beta, Gamma, Delta, and Omicron, where the Delta variant is thresholded by time. FIG. 15A shows delta sequences only from July-August 2021, and FIG. 15B shows delta sequences from November-December 2021. The sequences for the other variants are taken from any time period. When FIGS. 15A-15B are compared, Delta sequences collected during November-December 2021 are more distinctive than Delta sequences from July-August 2021. Similarly, FIG. 15C shows delta sequences only from April-May-June 2021, and FIG. 15D shows delta sequences only from December 2021. The sequences for the other variants are taken from any time period. When FIGS. 15A and 15C-15D are compared, Delta sequences collected during December 2021 are more distinctive than Delta sequences from April-May-June 2021 and Delta sequences from July-August 2021. Together, FIGS. 15A-15D show a rightward shift of the 9-mer distinctiveness overtime, suggesting that the metric is at least partially reflective of evolutionary time. FIG. 15D allows a head-to-head comparison of Omicron with Delta variant sequences that were circulating in the same time frame (December). As shown in FIG. 15D, Omicron sequences tend to have more distinctive 9-mers than contemporaneously collected delta sequences, indicating that Omicron may be more distinctive than would be expected from evolutionary time alone.

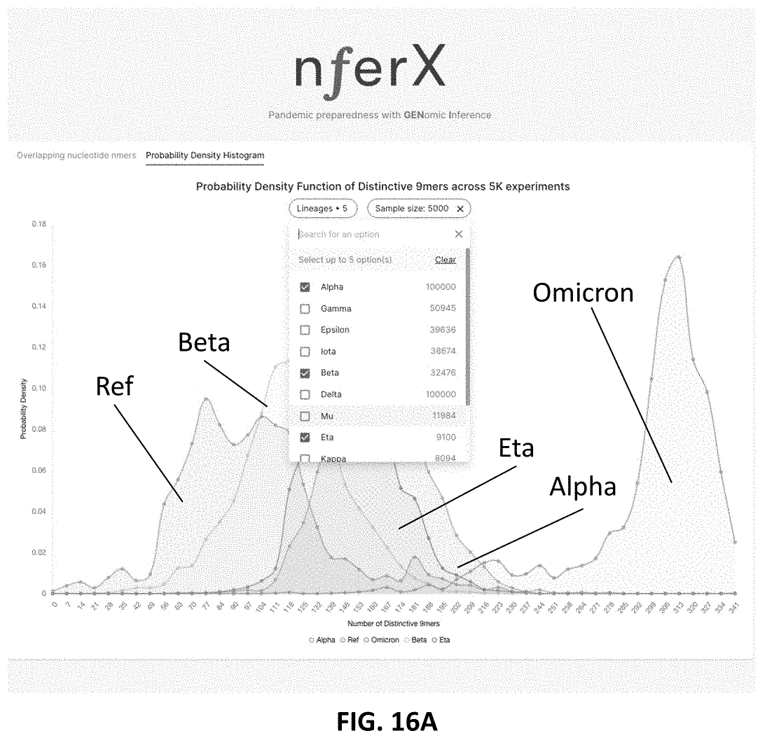

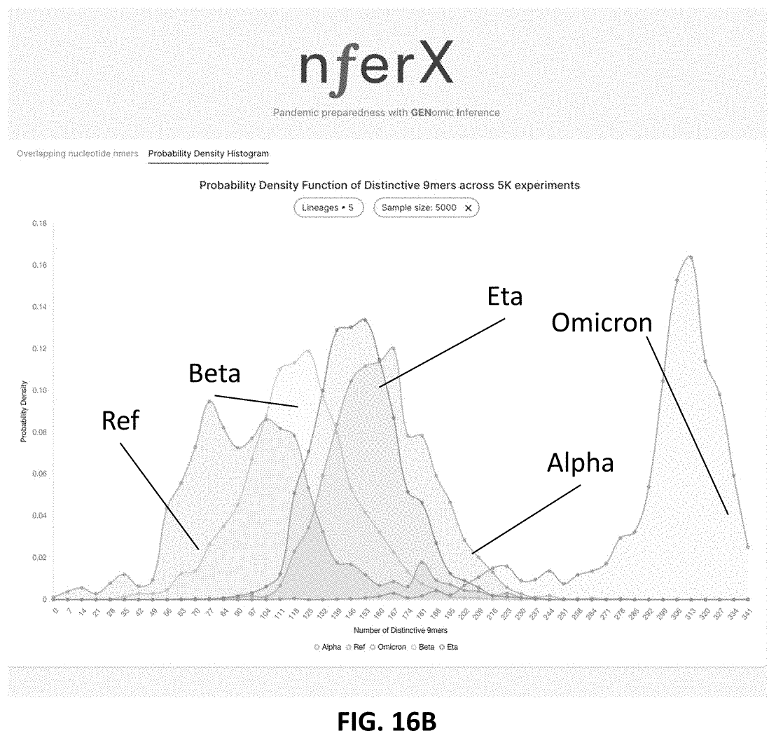

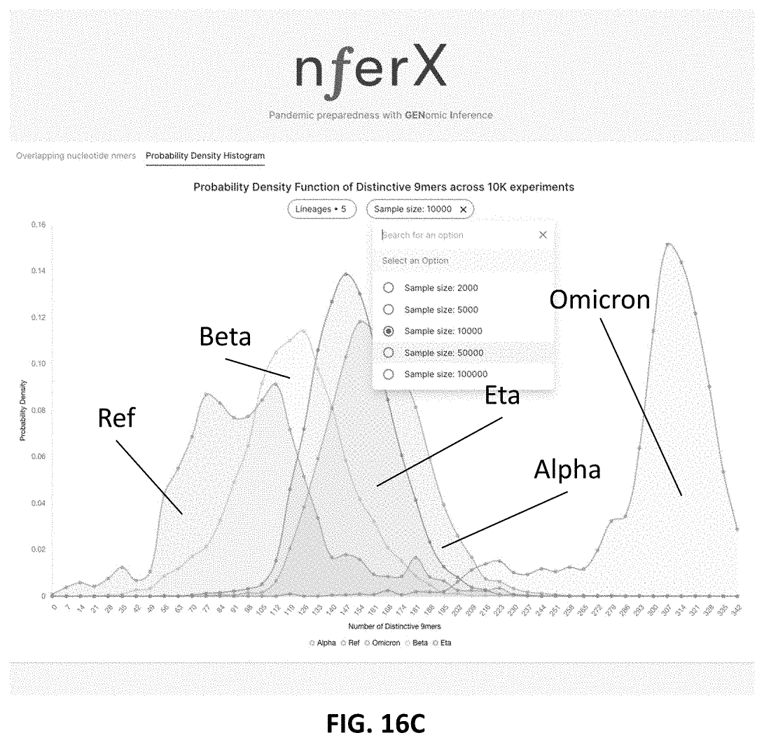

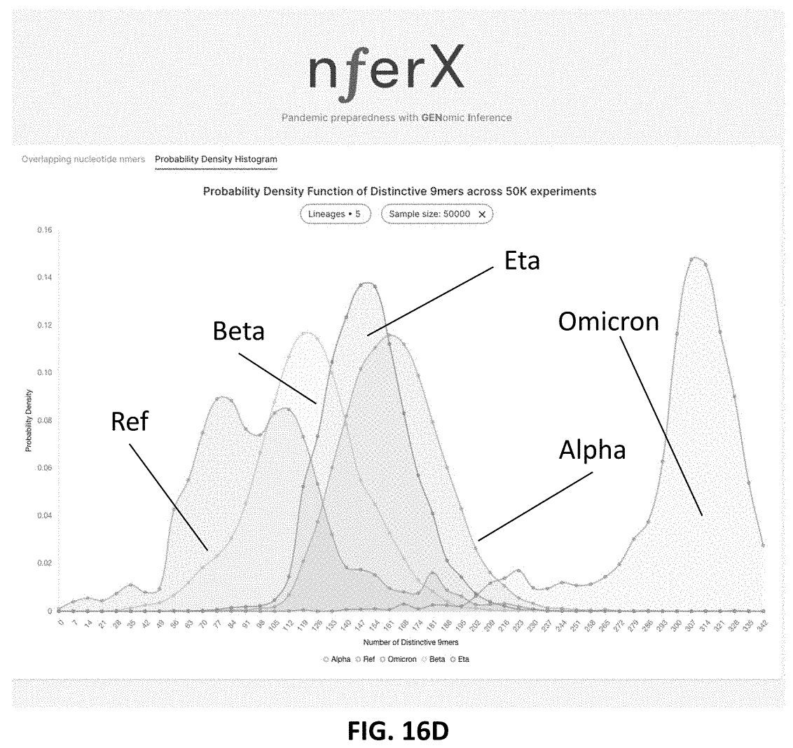

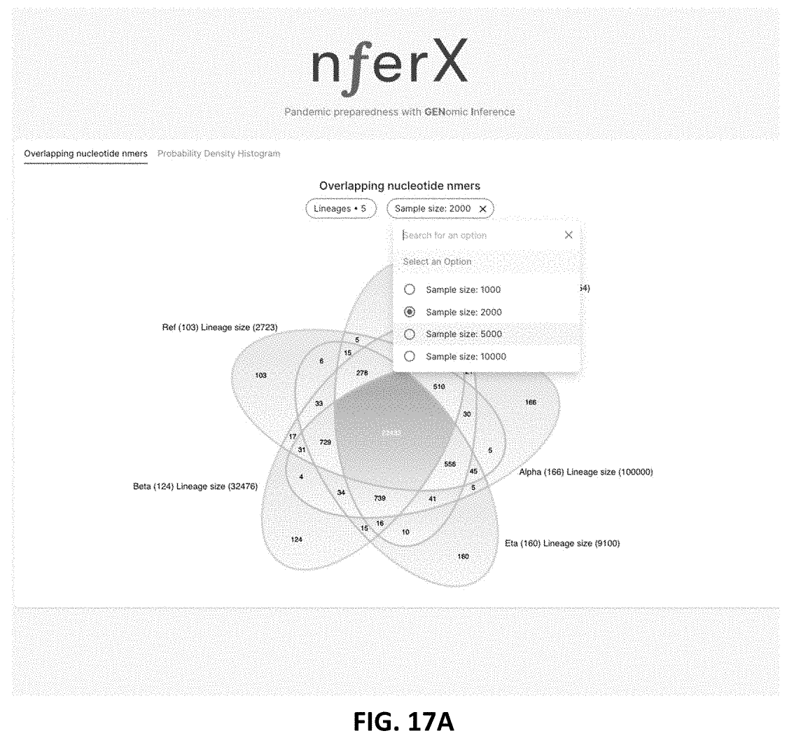

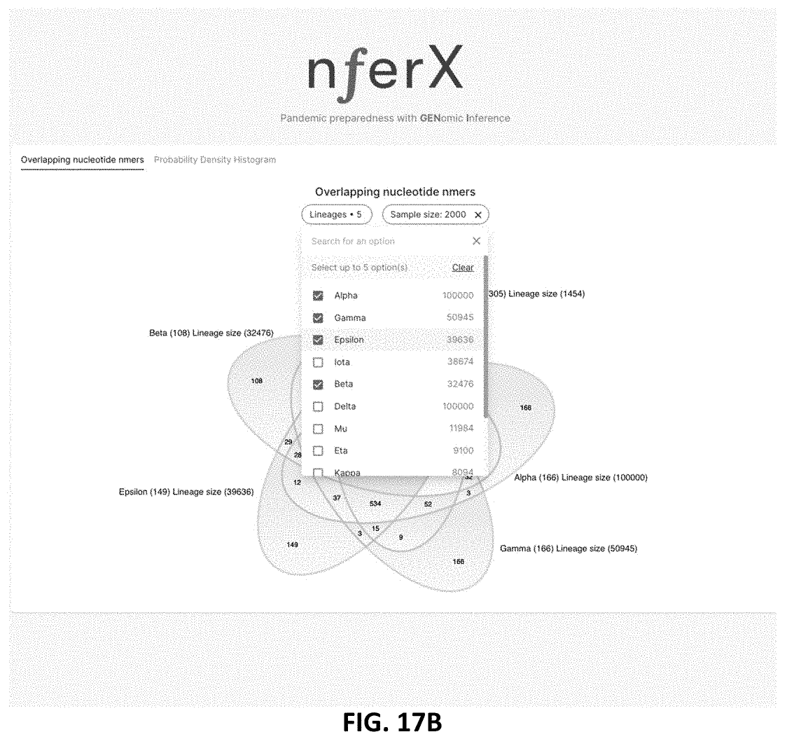

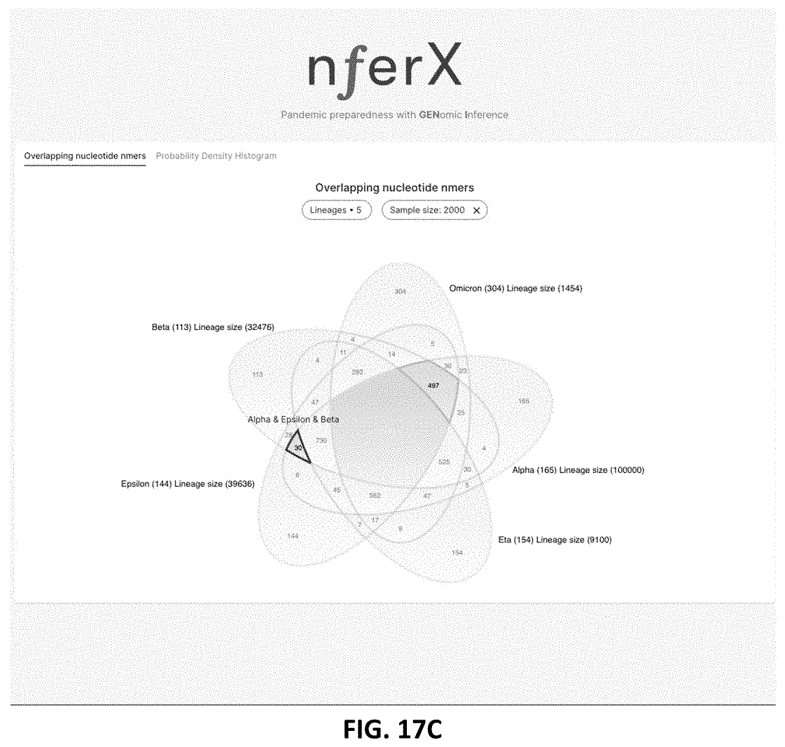

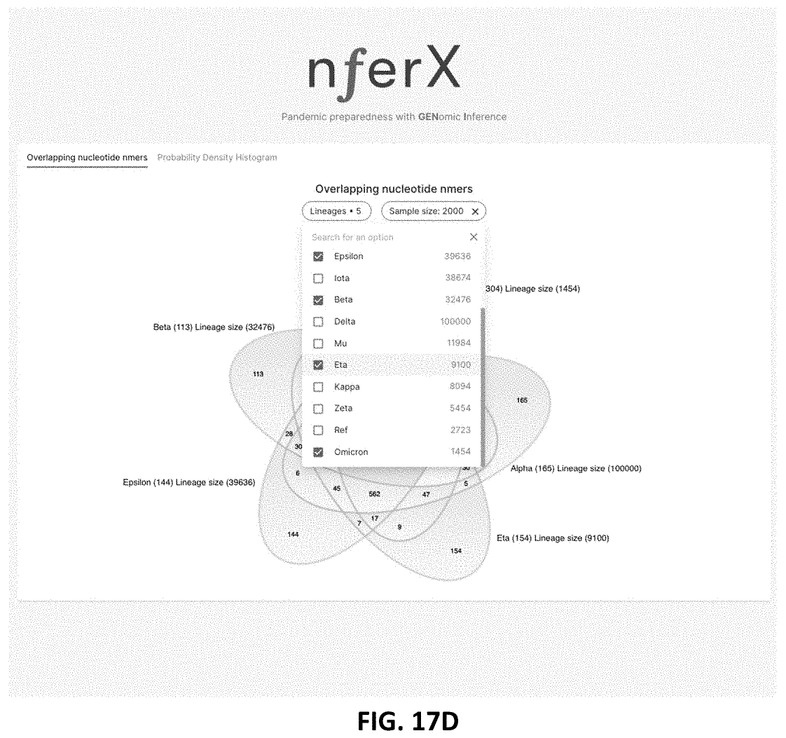

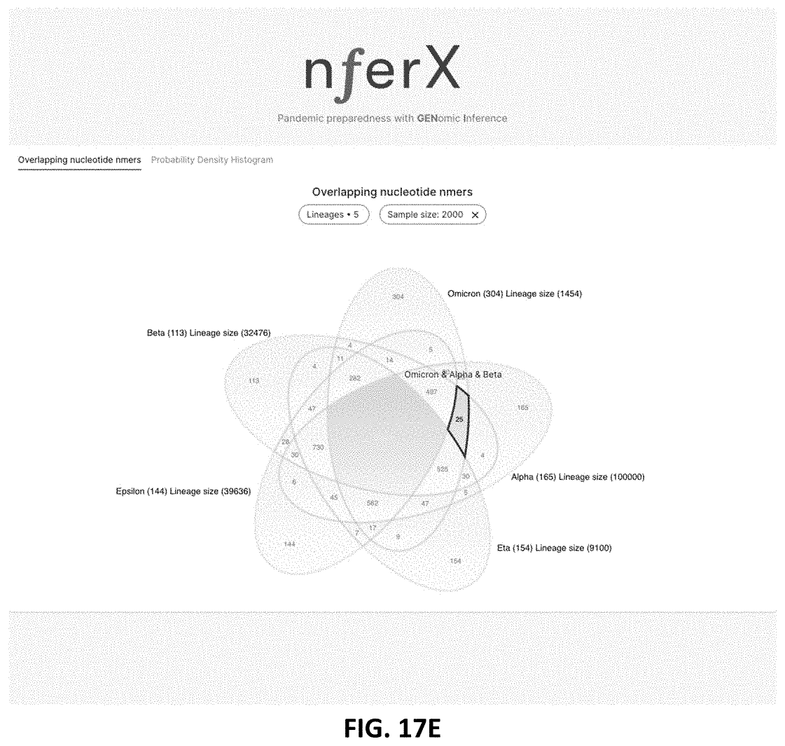

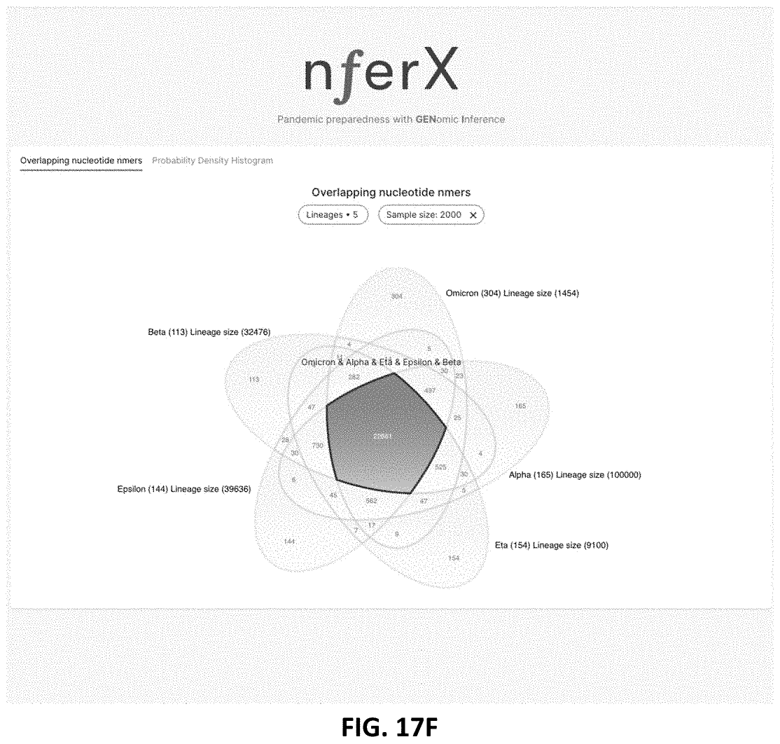

FIGS. 16A-16D and 17A-17F show screenshots of a user interface. FIGS. 136-16C show distributions of distinctive n-mers, and FIGS. 16A-16F show Venn diagrams of n-mers.

FIG. 16A shows a user selecting lineages from a dropdown menu to generate distributions of the number of distinctive n-mers. In FIG. 16B, the user has selected Alpha, Beta, and Eta and a sample size of 5000 (number of sets or combinations compared). FIG. 16B shows the resulting distributions. FIG. 16C shows a user selecting the sample size a dropdown menu to generate distributions of distinctive n-mers. The user has selected a sample size of 10,000. FIG. 16D shows a distribution with 50,000 samples. Note that some of the distributions shifted as more samples were added.

FIGS. 17A-17F show 5-way Venn diagrams for variants that show the number of n-mers in each region of the Venn Diagram. The Venn diagrams show the number of distinctive n-mers for each variant in the outer-most lobe of the Venn diagram. The number in parenthesis after the variant name is the number of distinctive n-mers. The lineage size is the number of sequences available for that variant. FIG. 17A shows a user selecting a sample size from a dropdown menu. FIG. 17B shows a user selecting the Alpha, Gamma, Beta, Epsilon, and Beta variants from a dropdown menu. FIG. 17C shows a highlighted region of a Venn diagram indicating the number of n-mers that overlap between the Alpha, Epsilon, and Beta variant (30 n-mers). FIG. 17D shows a user selecting the Epsilon, Beta, Eta, and Omicron variants from a dropdown menu. FIG. 17E shows a highlighted region of a Venn diagram indicating the number of n-mers that overlap between the Omicron, Alpha, and Beta variants (24 n-mers). FIG. 17F shows a highlighted region of the Venn diagram indicating the number of n-mers that overlap across all five variants (Beta, Omicron, Epsilon, Alpha, Eta).

Data was obtained from the following sources
1. Reference sequences, shown in Table 3.
   FASTA files for whole transcriptomes of different variants of concern.
   Sequences were downloaded from https://www.ncbi.nlm.nih.gov/nuccore/ using the command line tool.

TABLE 3
Sources of reference sequence data for variants of concern

| | | | NCBI |
|---|---|---|---|
| Variant Name | Lineage | WHO Designation | Accession ID |
| Wuhan | A | | NC_045512 |
| Alpha | B.1.1.7 | Variant of Concern | OL689430 |
| Beta | B.1.351 | Variant of Concern | OL691390 |
| Gamma | P.1 | Variant of Concern | OL615161 |
| Delta | B.1.617.2 | Variant of Concern | OL696821 |
| Omicron | B.1.1.529 | Variant of Concern | OL672836 |

2. GISAID
   a. https://www.gisaid.org/
   b. 5,953,706 sequences across 1,544 lineages

Aligned, Protein-based Analysis of Distinctiveness of New Sequences

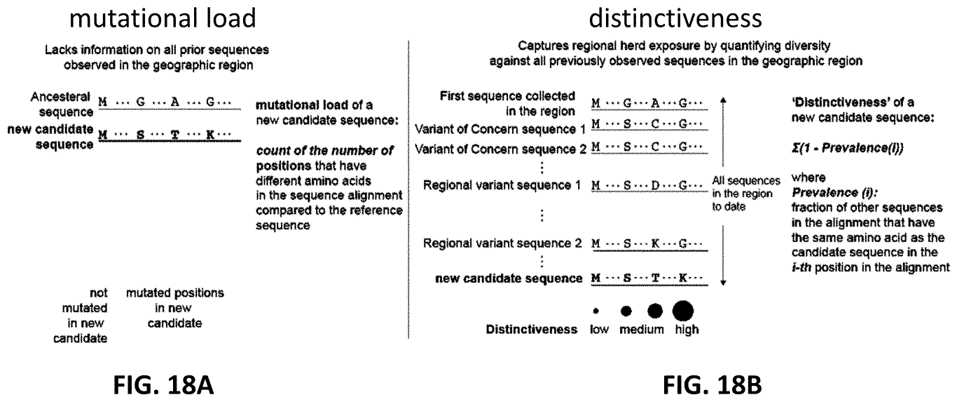

In the examples shown in FIGS. 18A-28, sequence ‘Distinctiveness’ of SARS-CoV-2 sequences was defined based on a proteome-wide comparison with all prior sequences from the same geographical region. In this example, protein sequences were aligned to a reference sequence. In this example a correlation was observed between sequence Distinctiveness relative to contemporary sequences and future change in prevalence of a newly circulating lineage (Pearson r=0.75), suggesting that the sequence Distinctiveness of emergent SARS-CoV-2 lineages is associated with their epidemiological fitness. It was further shown that the average sequence Distinctiveness of sequences belonging to a lineage, relative to the sequence Distinctiveness of other sequences that occur at the same place and time (n=944 location/time data points) is predictive of future increases in prevalence (AUC=0.88, 0.86-0.90 95% confidence interval). For example, by assessing the Delta variant in India versus Brazil, it was shown that the same lineage can have different sequence Distinctiveness-contributing positions in different geographical regions depending on the other variants that previously circulated in those regions. Overall, these examples shown that real-time assessment of new SARS-CoV-2 variants in the context of prior regional herd exposure via sequence Distinctiveness can augment genomic surveillance efforts.

In the examples shown in FIGS. 18A-28, the metric sequence ‘Distinctiveness’ can capture the proteome-level novelty of emerging SARS-CoV-2 sequences against documented regional lineages. Sequence Distinctiveness can quantify previous herd exposure to viral sequences that are similar to the current sequence, capturing an important factor of viral epidemiological fitness. This approach views viral evolution through a lens that considers the pressure to evolve new strains harboring protein content to which communities have not previously been exposed. The same lineage can have different sequence Distinctiveness values simultaneously in different countries, as well as different Distinctiveness-contributing positions. It was found that the relative sequence Distinctiveness of emergent SARS-CoV-2 lineages is associated with their epidemiological fitness, as defined by the change in the lineage prevalence.

Quantification of number of distinct positional amino acids for prevalent SARS-CoV-2 lineages: In this example, individual substitutions, insertions and deletions for each aligned SARS-CoV-2 protein sequence along with the corresponding PANGO designation were obtained from the GISAID (https://www.gisaid.org) database, on May 3, 2022. Only sequences labeled as “complete” and “high coverage” from the GISAID data were considered. These sequences were collected from 28 top sequencing countries (Table 4), and this resulted in a total of 4,926,906 sequences. For the original Wuhan strain and the five VOCs (Alpha, Beta, Gamma, Delta and Omicron), the PANGO classification was obtained from the CDC website (https://www.cdc.gov/coronavirus/2019-ncov/variants/variant-classifications.html).

TABLE 4
Number of Sequences per Geographic Region Included in Analysis

| Country | Sequences from Region |
|---|---|
| United Kingdom | 1,190,215 |
| Denmark | 379,340 |
| Germany | 290,861 |
| Japan | 182,846 |
| Canada | 175,571 |
| France | 121,274 |
| Sweden | 116,432 |
| Brazil | 84,477 |
| Switzerland | 80,524 |
| Turkey | 77,479 |
| Netherlands | 76,297 |
| Italy | 70,548 |
| Spain | 66,644 |
| India | 63,720 |
| Belgium | 57,909 |
| Norway | 45,103 |
| Slovenia | 40,782 |
| Australia | 36,864 |
| Poland | 35,956 |
| Ireland | 35,796 |
| Mexico | 33,546 |
| Finland | 24,028 |
| South Korea | 23,891 |
| Lithuania | 20,516 |
| Portugal | 19,349 |
| Israel | 17,106 |
| South Africa | 11,362 |

Calculation of sequence Distinctiveness: In this example, for a given sequence, sequence Distinctiveness within a geographical region of interest (e.g., a country) can be defined as the average distances at the amino-acid level between that sequence and all prior sequences that were collected at least one calendar day before that sequence. The time period may limited by the time-resolution of the data. For example, for a sequence, s, its sequence Distinctiveness, D(s), is calculated using the following formula:

$$D ( s ) = \frac{1}{{N}_{p}} {\sum }_{{s}^{′} ∈ prior sequences} {\sum }_{p = 1}^{AAA} 1 - δ ( s ( p ) - {s}^{′} ( p ) )$$

Where Nₚ is the number of prior sequences, s′ is one specific prior sequence, the inner sum is over all pairwise aligned amino acid positions, and δ(s(p)−s′(p)) evaluates to 1 if sequences and s′ have the same amino-acid identity (one of twenty amino acids, a deletion, or a specific insertion) at position p and 0 otherwise. In this example, positions of amino acids are determined relative to the Wuhan-Hu-1 reference, and insertions were treated as a single modification at the site of insertion. In cases where a nonsense mutation occurred, resulting in an early stop codon, mutations that followed this stop codon were not considered.

Calculation of sequence mutational load: The mutational load was calculated as the number of mutations away from the ancestral Wuhan-Hu-1 sequence. Similar to in the sequence Distinctiveness calculation, insertions were counted as a single mutation. In cases where a nonsense mutation occurred, resulting in an early stop codon, mutations that followed this stop codon were not considered.

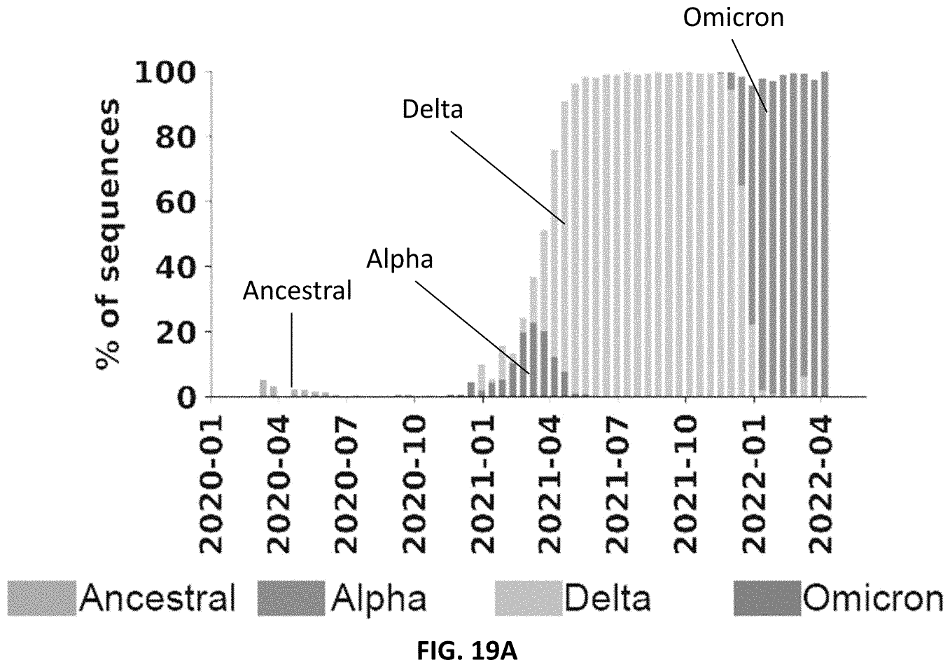

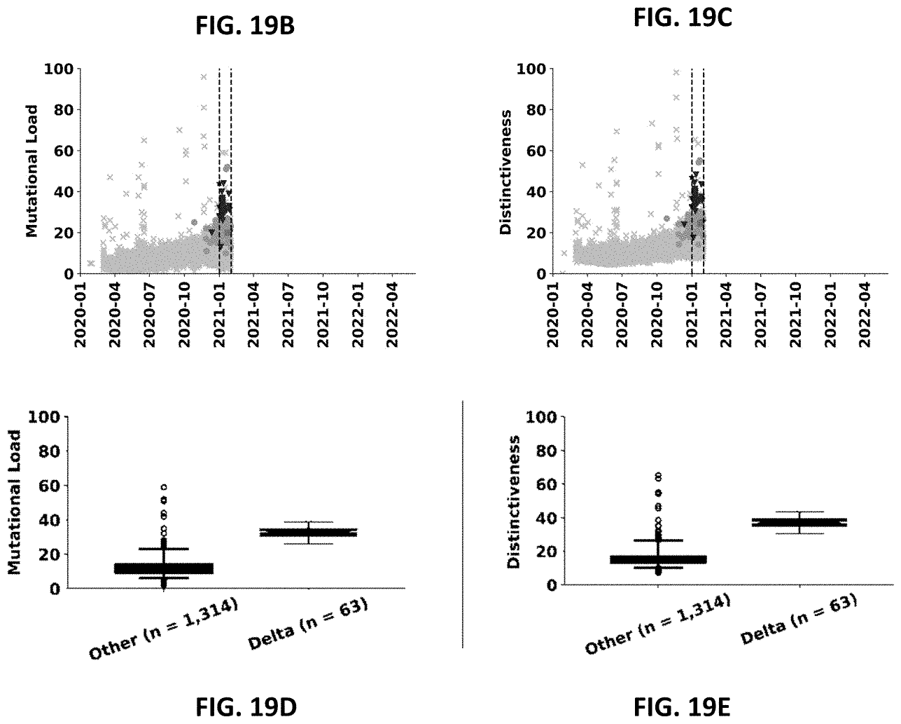

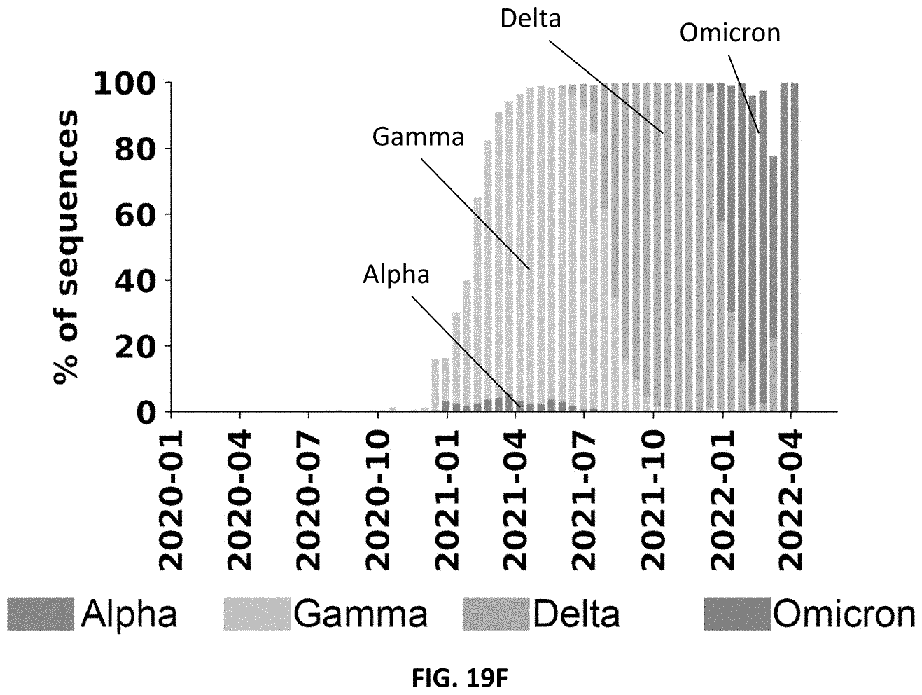

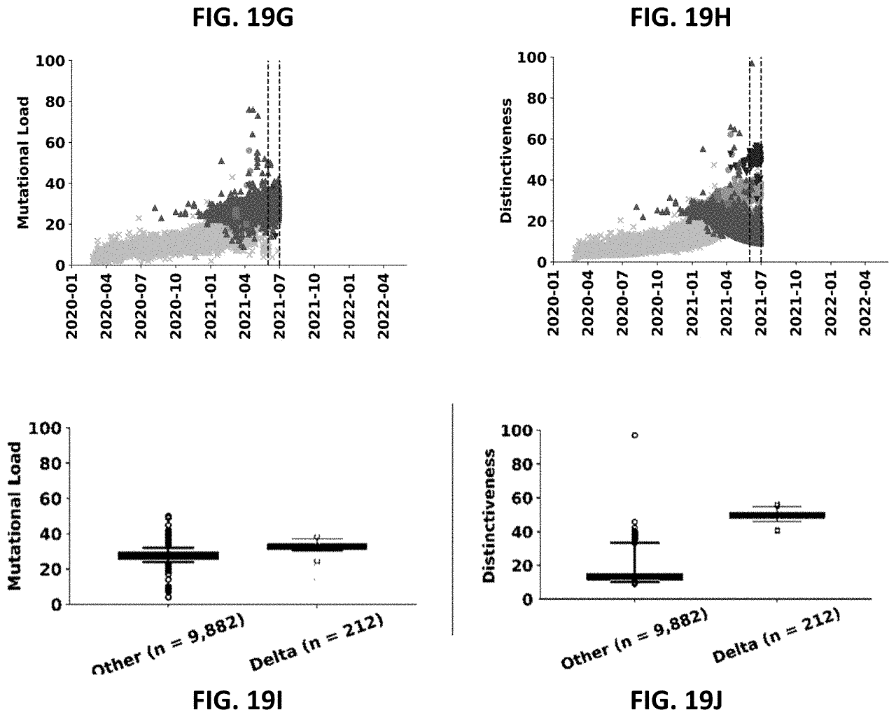

Calculating local prevalence of variants of concern: The local prevalence of a SARS-CoV-2 variant, as reported in FIGS. 19A and 19F was calculated as the percentage of SARS-CoV-2 sequences in GISAID that were assigned to a lineage comprising that variant, during specific time windows and in specific countries.

Correlating the Distinctiveness and changes in future prevalence of SARS-CoV-2 lineages: The average sequence Distinctiveness of sequences in a set during a 28 day window was correlated to the change in prevalence of the corresponding set, defined as prevalence (t+56 to t+84)—prevalence (t to t+28), where t denotes time. For the analysis in FIGS. 24A-24B, data points are shown only for time periods in which one of the VOCs (Alpha, Beta, Gamma, Delta, and Omicron) first reached >5% prevalence in a given geographic region (defined as a country or US state); all variants present in the geographical region at included time windows are shown. This results in 944 data points, spanning 154 time windows in 78 geographical regions. An alternate version of this analysis, with inclusion of all available time windows (36,000 time windows spanning the same 78 geographical regions) is shown in FIGS. 25A-25B and yields similar conclusions as those described in the main text.

Receiver operator characteristic (ROC) curves were generated from these data using Scikit-learn, using binary labels based on a minimum 20 percentage point increase in lineage prevalence for a country/time datapoint. Resulting area under the curve (AUC) and threshold values, maximizing the sum of Sensitivity and Specificity, were found to be robust with respect to the cut-off used for labeling the data based on the percentage point increase (FIGS. 26A-26B). Bootstrap resampling (10,000 samples) of the underlying data points (scatter points in FIG. 24A) was used to estimate 95% confidence intervals on the resulting AUC values.

Capturing emerging SARS-CoV-2 Using Sequence Distinctiveness: Sequence distinctiveness can be computed at the global level or at a regional level for any chosen time period. The sequence Distinctiveness of the VOCs was compared with contemporary sequences and the relationship between Distinctiveness of a sequence and the change in its regional prevalence was investigated.

For comparison, the ‘Mutational load’ of the same sequences were also reported. Mutational load is simply the number of mutations in the new sequence compared with the ancestral reference sequence (GenBank: MN908947.3), e.g., a single reference sequence, and as such it does not account for the entirety of SARS-CoV-2 evolution or the local prevalence of sequences. FIGS. 18A-18B show a comparison of mutational load and sequence Distinctiveness.

As shown, in FIG. 18A, mutational load compares a new candidate sequence with one ancestor sequence. The candidate sequence is aligned with the ancestral sequence, and each amino acid of the candidate is compared against the amino acid at the same position in the ancestral sequence. The mutational load of the candidate sequence is a count of the number of positions that have different amino acids in the sequence alignment compared to the reference ancestral sequence. Thus, the mutational load lacks information on all prior sequences observed in a geographic region.

In contrast, as shown in FIG. 18B, sequence Distinctiveness can capture regional herd exposure by quantifying diversity against all previously observed sequences in a geographic region. Sequence distinctiveness can be determined by comparing a new candidate sequence with all prior sequences in the region (e.g., the first sequence, different variants of concern, and different regional variants). The Distinctiveness of a sequence can be calculated by calculating the sum of (1−prevalence), where prevalence(i) for a given position is the fraction of other sequences in the alignment that have the same amino acid as the candidate sequence in the i^{th} position. As shown in FIG. 18B, for the four amino acids in the illustrative candidate sequence, each position has a different Distinctiveness. The first position has a low Distinctiveness because it is the same as the amino acids in the same position of the prior sequences. The fourth position has a high Distinctiveness because it is different from all of the amino acids in the same position of the prior sequences. The second and third positions have medium Distinctiveness.

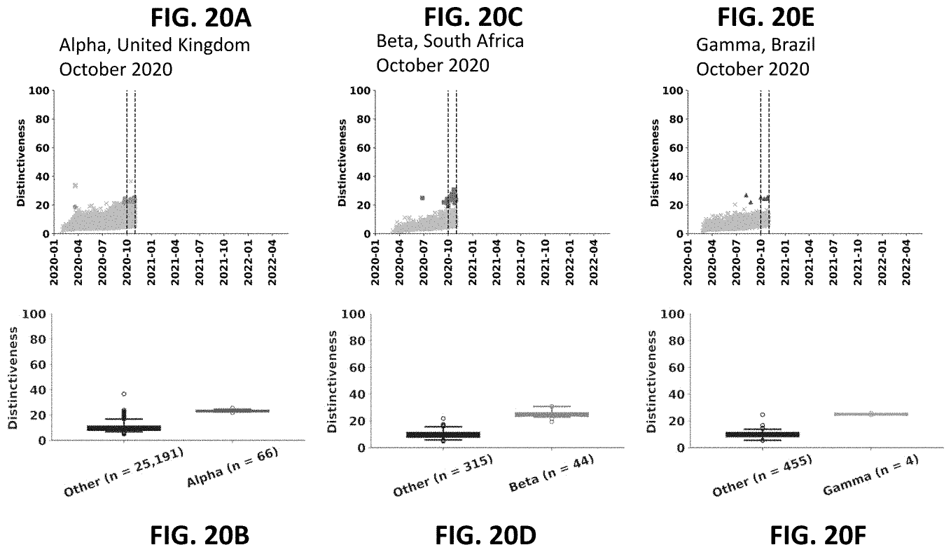

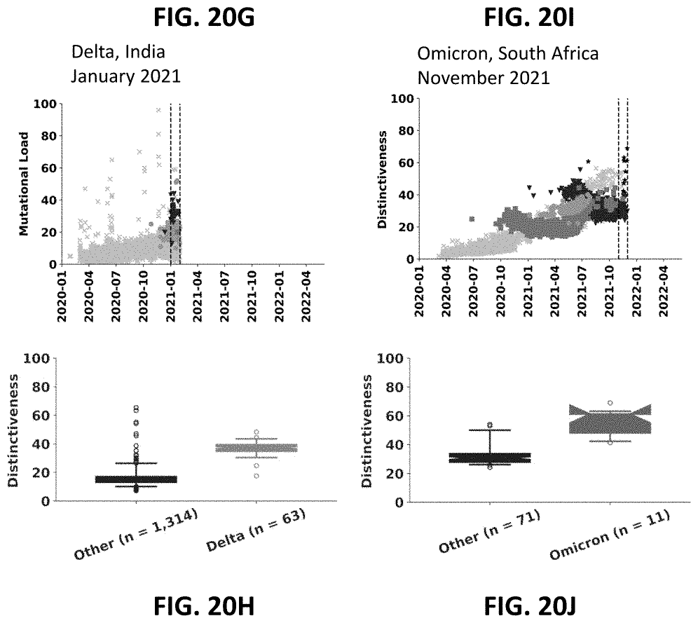

Mutational load and sequence Distinctiveness were computed for the time period during the emergence of the VOCs in the country of their emergence. As shown in FIGS. 19A-J, FIGS. 20A-J, and 21A-21J, both mutational load and Distinctiveness values of VOC sequences were significantly higher than contemporary lineages at the time that they emerged.

FIGS. 19A-J show a comparison of mutational load and Distinctiveness of VOCs during the emergence of the variants in India and Brazil. For example, FIGS. 19A-19E show the emergence of the Delta variant in India during January 2021. FIG. 19A shows the prevalence of different VOCs in India as a function of time. FIG. 19B shows mutational load for different variants in India over time, while FIG. 19C shows sequence Distinctiveness for different variants in India over time. Different VOCs are shown with different shaped markers (Alpha: circles, Beta: squares, Gamma: upward triangle, Delta: downward triangle, Omicron: star) and other sequences are shown with an x. FIG. 19D shows mutational load for Delta sequences compared to other sequences in India in January 2021, while FIG. 19E shows sequence Distinctiveness of Delta compared to other sequences in India in January 2021. As shown in FIGS. 19B-19E, both mutational load and sequence Distinctiveness of the Delta variant in India were significantly higher than that of the other contemporary lineages. This raises the question of whether Delta variant sequences were also competitive in other countries.

FIGS. 19F-19J show the emergence of variants in June 2021 in Brazil, where the Gamma variant was dominant prior to the arrival of the Delta variant. FIG. 19F shows the prevalence of different VOCs in Brazil as a function of time. FIG. 19G shows mutational load for different variants in Brazil over time, while FIG. 19H shows sequence Distinctiveness in Brazil for different variants over time. Different VOCs are shown with different shaped markers (Alpha: circles, Beta: squares, Gamma: upward triangle, Delta: downward triangle, Omicron: star) and other sequences are shown with an x. FIG. 19I shows mutational load for Delta sequences compared to other sequences in Brazil in June 2021, while FIG. 19J shows sequence Distinctiveness of Delta compared to other sequences in Brazil in June 2021. As shown in FIGS. 19G-19J, while the mutational load of the Delta variant was comparable to those of contemporary lineages in Brazil, the sequence Distinctiveness of the Delta variant was significantly higher. Indeed, as shown in FIG. 19F, the Delta variant outcompeted the Gamma variant to become the dominant strain in Brazil.

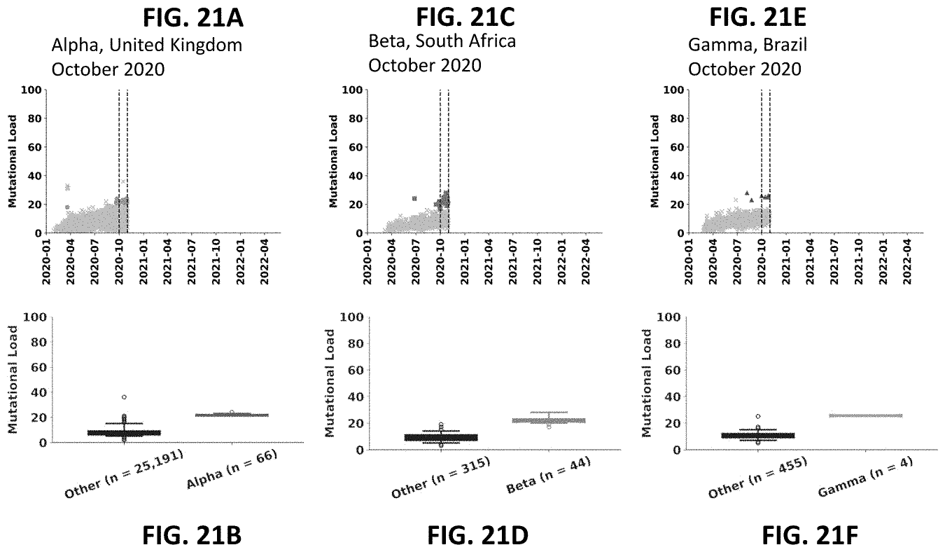

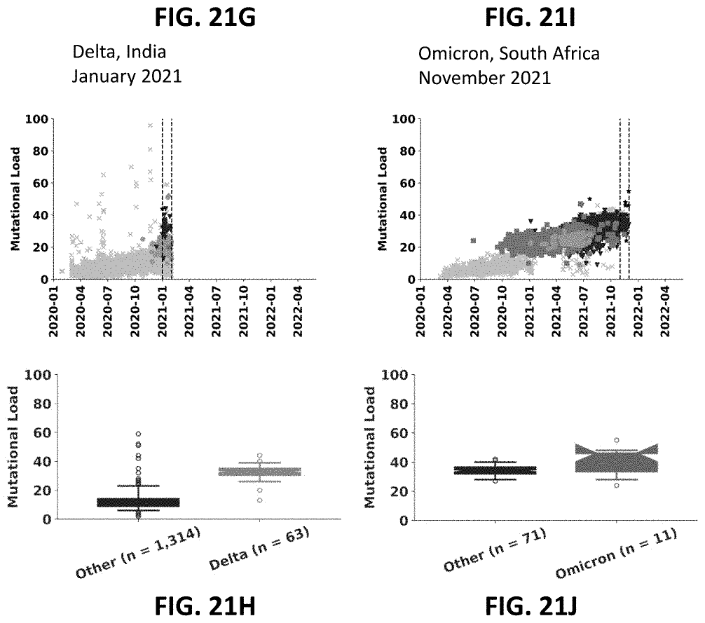

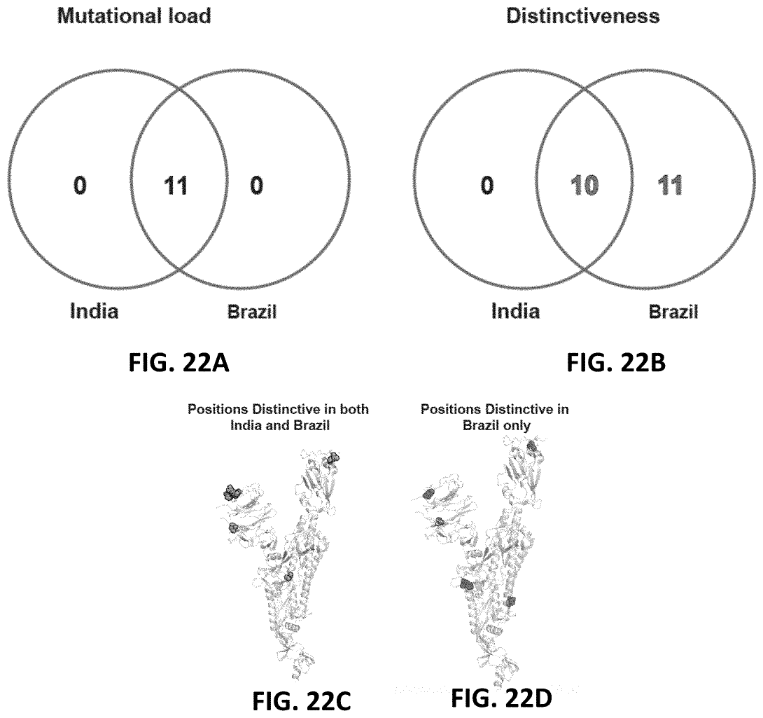

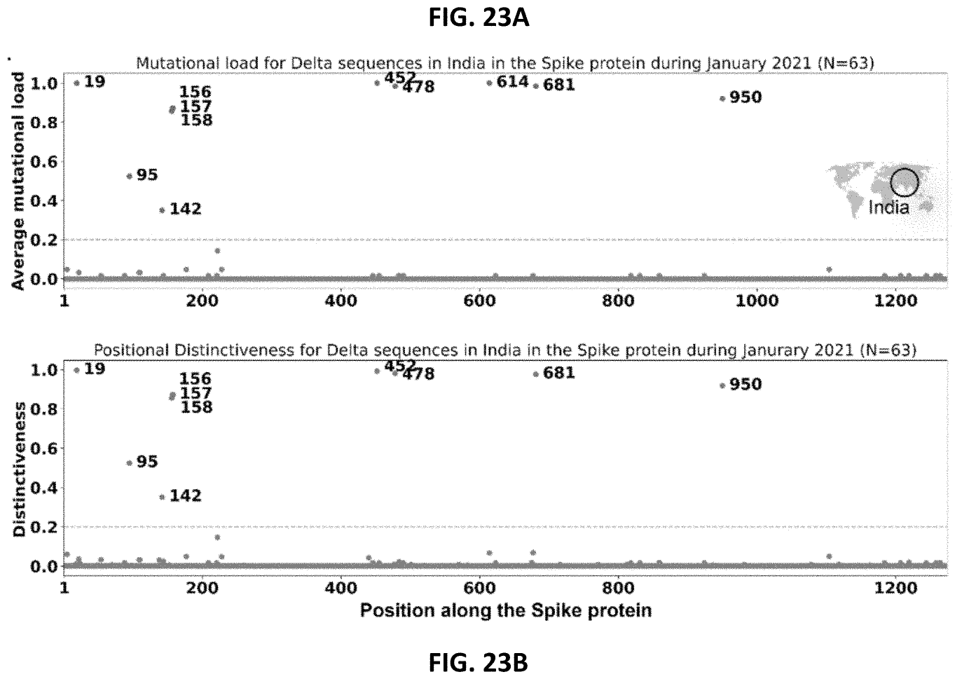

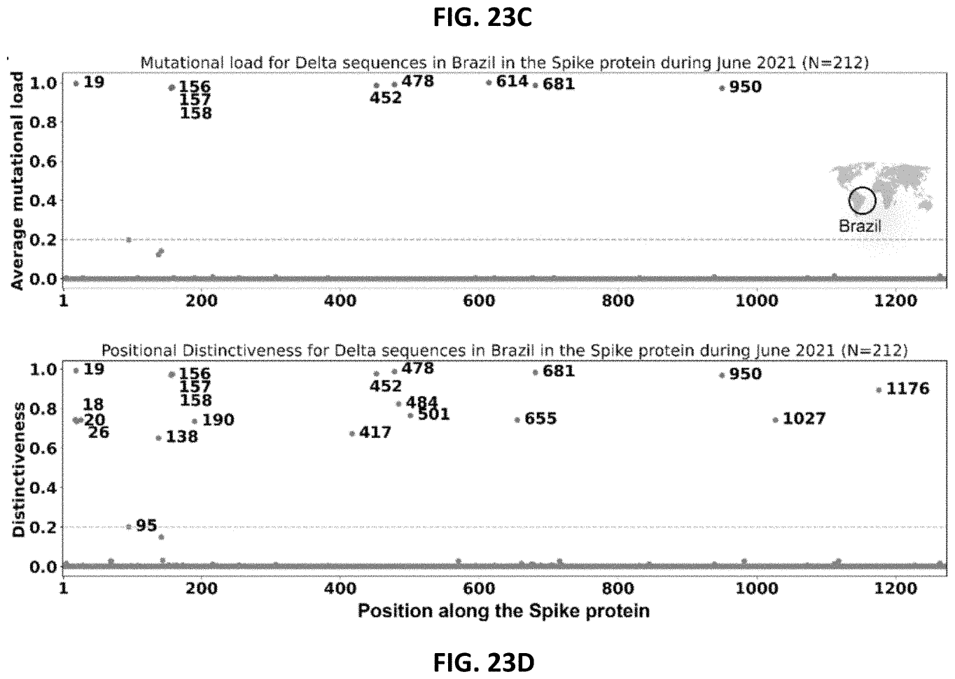

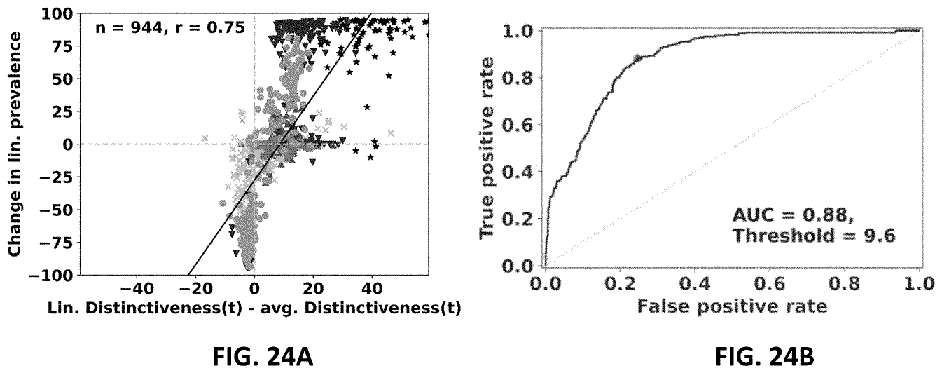

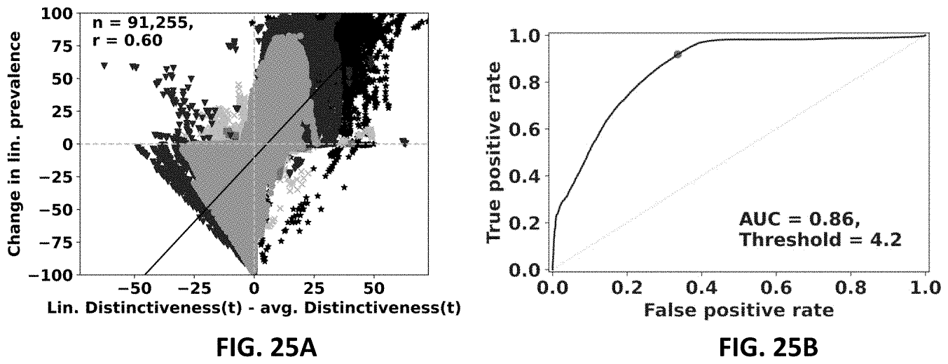

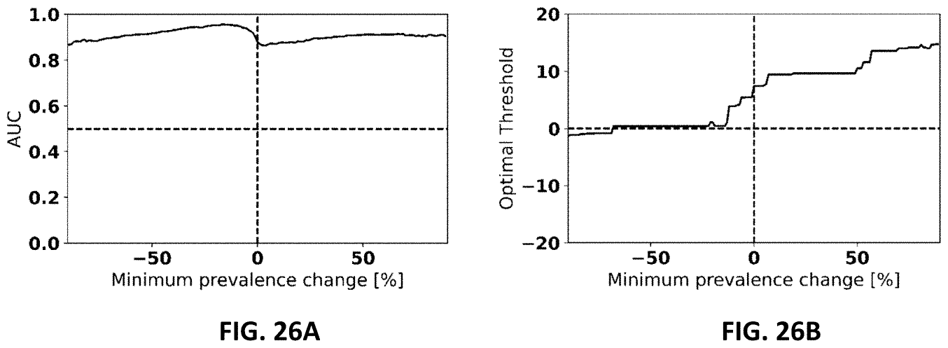

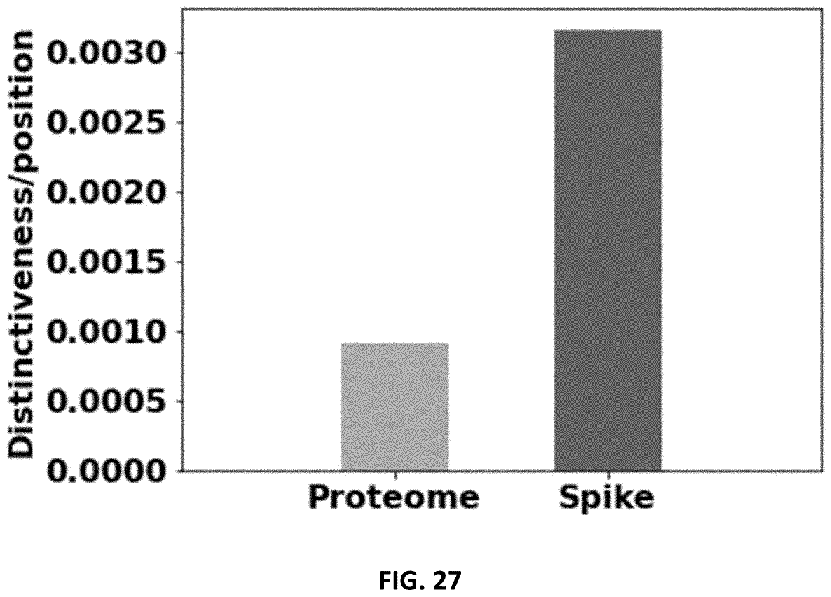

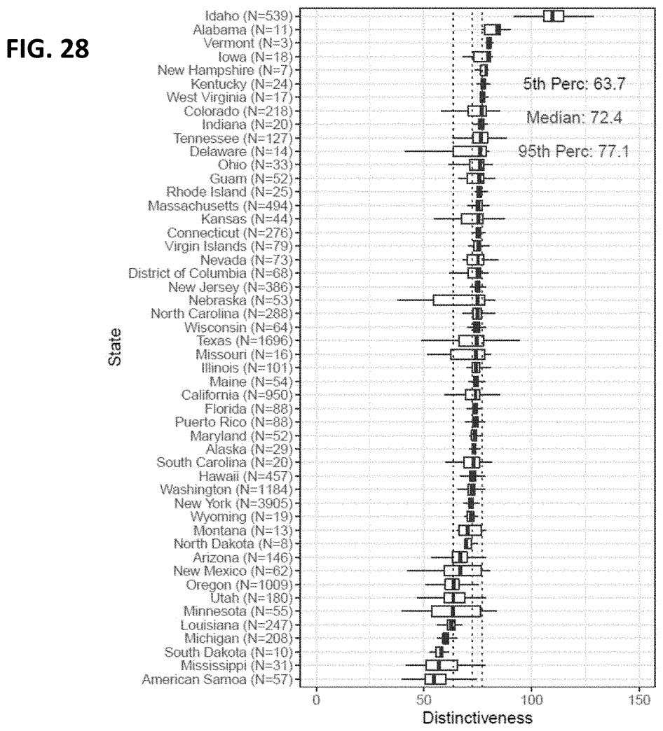

FIGS. 20A-20J show Distinctiveness of variants of concern during the time when they first appeared. Different VOCs are shown with different shaped markers (Alpha: circles, Beta: squares, Gamma: upward triangle, Delta: downward triangle, Omicron: star) and other sequences are shown with an x. FIGS. 20A-20B show the sequence Distinctiveness of the Alpha variant compared to other sequences in the United Kingdom in October 2020. FIGS. 20C-20D show the sequence Distinctiveness of the Beta variant in South Africa in October 2020. FIGS. 20E-20F show the sequence Distinctiveness of the Gamma variant in Brazil in October 2020. FIGS. 20G-20H show the sequence Distinctiveness of the Delta variant in India in January 2021. FIGS. 201-20J show the sequence Distinctiveness of the Omicron variant in South Africa in November 2021. In all cases, the sequence Distinctiveness of the VOCs is significantly higher (p-value<0.001) than that of contemporary sequences. For Alpha, Beta, and Gamma, sequence Distinctiveness values of sequences collected during October 2020 are shown; for Delta sequence Distinctiveness values of sequences collected during January 2021 are shown; and for Omicron sequence Distinctiveness values of sequences collected during November 2021 are shown.

FIGS. 21A-21J show the mutational load of variants of concern during the time when they first appeared. Different VOCs are shown with different shaped markers (Alpha: circles, Beta: squares, Gamma: upward triangle, Delta: downward triangle, Omicron: star) and other sequences are shown with an x. FIGS. 21A-21B show the mutational load of the Alpha variant compared to other sequences in the United Kingdom in October 2020. FIGS. 21C-21D show the mutational load of the Beta variant in South Africa in October 2020. FIGS. 21E-21F show the mutational load of the Gamma variant in Brazil in October 2020. FIGS. 21G-21H show the mutational load of the Delta variant in India in January 2021. FIGS. 21I-21J show the mutational load of the Omicron variant in South Africa in November 2021. In all cases, the mutational load of the VOCs is significantly higher (p-value<0.001) than that of contemporary sequences. For Alpha, Beta, and Gamma, Mutational load values of sequences collected during October 2020 are shown; for Delta Mutational load values of sequences collected during January 2021 are shown; and for Omicron Mutational load values of sequences collected during November 2021 are shown.

Next, the specific positions that contribute most to the observed sequence Distinctiveness values of the Delta variant in India and Brazil were assessed. The mutational frequency and average Distinctiveness contribution were compared for each amino acid position in the Spike protein of Delta variant sequences collected in India versus Brazil. These comparisons are shown in FIGS. 22A-22D and 23A-23D. FIGS. 22A-22B show Venn diagrams comparing the positions that contribute to Mutational load (FIG. 22A) or high Distinctiveness (FIG. 22B). While sequences from India and Brazil had the same mutational load (11) (FIG. 22A), sequences from Brazil had 11 additional positions with distinctiveness that were not present in India (FIG. 22B). While mutational load compares each sequence to an ancestral sequence (11 mutations for Brazil), distinctiveness compares each sequence to prevalent sequences in the region (21 distinctive positions in Brazil). FIGS. 22C-22D show the positions that have high Distinctiveness highlighted on the protein structures of the Spike protein as spheres (PDB identifier: 6VSB). FIG. 22C shows positions that are distinctive in both India and Brazil, while FIG. 22D shows positions that are distinctive in Brazil only. These regional differences reflect that, in Brazil, the comparison was generally between Delta and Gamma (the previously prevalent variant in Brazil), whereas in India, the comparison was generally between Delta and Alpha (the previously prevalent variant in India). Thus, distinctiveness can be used to compare new sequences in a region with sequences of variants that were recently prevalent in that region.

FIGS. 23A-23D show position distinctiveness, the contribution of Distinctiveness for a given position, for spike protein amino acid sequences. FIGS. 23A-23B show the mutational load (FIG. 23A) and distinctiveness (FIG. 23B) as a function of position along the spike protein for Delta sequences in India during January 2021. FIGS. 23C-23D show the mutational load (FIG. 23C) and distinctiveness (FIG. 23D) as a function of position along the spike protein for Delta sequences in Brazil during June 2021. The x-axes denote the amino acid positions in the spike protein and the y-axes denote the average mutational load (FIGS. 23A and 23C) or the Distinctiveness (FIGS. 23B and 23D). Horizontal lines at y=0.2 denote a high threshold above which amino acid positions are labeled.

In India, where the Delta variant originated, the 11 mutated positions correspond almost exactly to the Distinctiveness-contributing positions. The exception is the 614 position on the Spike protein. This position has not contributed to the Delta variant's Distinctiveness as it has been highly prevalent globally (e.g., present in over 99% of SARS-CoV-2 genomes deposited in GISAID) since June 2020. Brazil, on the other hand, experienced a large wave of cases dominated by the Gamma variant before the arrival of the Delta variant. Here, as shown in FIG. 22C, in addition to the same 10 Spike protein mutations that were observed in India (FIGS. 23A-23B), there were 11 other positions that further contributed to Brazil's regional Distinctiveness (FIGS. 23C-23D). These additional positions correspond to known Gamma lineage-defining mutations (L18F, T20N, P26S, D138Y, R1905, K417T, E484K, N501Y, H655Y, T10271, V1176F). These differences between India and Brazil show that sequence Distinctiveness can capture how new sequences (for example, Delta in the examples shown in FIGS. 23A-23D), differ from sequences that previously circulated in a region (for example, Gamma for Brazil and Alpha for India in the examples shown in FIGS. 23A-23D). For example, because many individuals in Brazil so far had been infected by the Gamma variant at the time that Delta emerged, differences at different positions between Gamma and Delta are relevant because, for example, they may allow the virus to avoid immune response due to previous Gamma exposure.

Association of Distinctiveness of Emergent Lineages with Epidemiological Fitness: In order to examine a possible relationship between sequence Distinctiveness and epidemiological fitness of SARS-CoV-2 lineages, the correlation between sequence Distinctiveness and change in prevalence for all circulating lineages (grouped as the VOCs and a single group combining all non-VOCs) was assessed in 78 geographical regions (27 countries and 51 US states).

FIGS. 24A-24B compare the correlations of sequence Distinctiveness of a lineage with its competitiveness in the geographic region (across 294 country/time data points). For the analysis in FIGS. 24A-24B, data points are shown only for time periods in which one of the VOCs (Alpha, Beta, Gamma, Delta, and Omicron) first reached >5% prevalence in a given geographic region (defined as a country or US state). FIG. 24A shows the change in prevalence as a function of distinctiveness of each lineage relative to the average distinctiveness of all sequences collected in the region at the same time (Lin. Distinctiveness(t)—avg. Distinctiveness(t)). Different VOCs are shown with different shaped markers (Alpha: circles, Beta: squares, Gamma: upward triangle, Delta: downward triangle, Omicron: star) and other sequences are shown with an x. As shown in FIG. 24A, sequence distinctiveness of a lineage, relative to the average sequence distinctiveness is predictive of future changes in prevalence (n=944, r=0.75). FIG. 24B shows the receiver operator curve (ROC) for predicting an increase in prevalence of greater than 20 percentage points from an initial 28-day time window and a subsequent 28-day time window, starting 56 days in the future.

FIGS. 25A-25B show the correlation between local sequence Distinctiveness of a lineage and the future change in prevalence of that lineage across all time periods. In comparison to FIGS. 24A-24B, FIGS. 25A-25B show data from all available time windows. FIG. 25A shows the correlation between change in prevalence of a lineage, from the current time to 56 days in the future, without any filtering of time periods, and the sequence distinctiveness of each lineage relative to the average sequence distinctiveness (465 time periods from 78 geographical regions). FIG. 25B shows the ROC for predicting an increase in prevalence of greater than 20 percentage points from an initial 28-day time window and a subsequent 28-day time window, starting 56 days in the future.

FIGS. 26A-26B show sensitivity analysis for the prediction of future changes in prevalence from average lineage sequence Distinctiveness. FIG. 26A shows the AUC for the prediction of lineages with increase in prevalence 56 days in the future from the average sequence Distinctiveness of sequences belonging to that lineage. The minimum increase in prevalence, used to define positive labels, was varied (x-axis), and AUC values were calculated (y-axis). FIG. 26B shows optimal threshold values for a positive prediction, using average lineage Distinctiveness relative to contemporary sequences as the predictive variable, that yield the highest sum of Specificity and Sensitivity.

As shown in FIGS. 24A and 25A, the relative sequence Distinctiveness of emergent SARS-CoV-2 lineages was associated with their change in lineage prevalence over eight weeks (Pearson r=0.75). In comparison, mutational load was found to have a lower association with change in prevalence (Pearson r=0.53). Further, as shown in FIGS. 24B and 25B, the average sequence Distinctiveness of a lineage in a country/time window can predict future increases in prevalence (AUC=0.88 [0.86-0.90, 95% CI], for predicting a greater than 20 percentage point increase in local prevalence).

Contribution of Spike Proteins to Distinctiveness: Since sequence Distinctiveness is intended to capture the fitness of a sequence in the context of previous herd exposure to similar sequences, Distinctiveness was investigated in the context of known immunogenic positions. The Distinctiveness of only Spike protein positions is shown in FIG. 27 in comparison with Distinctiveness of positions of the full proteome. As shown in FIG. 27, the Spike positions (average Distinctiveness of 0.007 at each position) exhibit increased contributions to overall Distinctiveness (average Distinctiveness of 0.003 at each position).

State-level analysis of Distinctiveness: FIG. 28 shows analysis of sequence Distinctiveness at the US state level. Table 5 shows the number of sequences from each state. As shown in FIG. 28, there is diversity in sequence Distinctiveness at the state level, including a high sequence Distinctiveness in Idaho. These differences indicate the usefulness of analysis of sub-regional sequence Distinctiveness to identify new variants.

TABLE 5
Number of Sequences per State Included in Analysis

| State | Sequences from Region |
|---|---|
| California | 266,055 |
| Texas | 111,293 |
| New York | 88,607 |
| Florida | 81,862 |
| Colorado | 71,456 |
| Massachusetts | 63,438 |
| Minnesota | 59,239 |
| Washington | 50,713 |
| Illinois | 42,502 |
| North Carolina | 41,688 |
| Michigan | 41,266 |
| Utah | 40,007 |
| Pennsylvania | 36,626 |
| Arizona | 33,575 |
| Wisconsin | 32,383 |
| New Jersey | 31,301 |
| Georgia | 30,652 |
| Maryland | 29,086 |
| Ohio | 26,979 |
| Oregon | 23,820 |
| Tennessee | 23,401 |
| Indiana | 22,777 |
| Virginia | 22,199 |
| Connecticut | 18,117 |
| Nevada | 17,858 |
| New Mexico | 17,512 |
| West Virginia | 17045 |
| Louisiana | 15,156 |
| Idaho | 14,290 |
| Missouri | 13,778 |
| Kentucky | 13,775 |
| South Carolina | 12,508 |
| Kansas | 11,199 |
| Alabama | 10,947 |
| Iowa | 10,727 |
| Wyoming | 10,454 |
| Nebraska | 10,184 |
| Maine | 9,355 |
| Montana | 9,069 |
| Rhode Island | 8,010 |
| Hawaii | 7,889 |
| Vermont | 7,493 |
| Arkansas | 7,044 |
| Delaware | 6,137 |
| New Hampshire | 5,942 |
| Mississippi | 5,781 |
| Alaska | 4,913 |
| North Dakota | 3,384 |
| District of Columbia | 3,192 |
| Oklahoma | 3025 |
| South Dakota | 2,761 |

It will be appreciated that while one or more particular materials or steps have been shown and described for purposes of explanation, the materials or steps may be varied in certain respects, or materials or steps may be combined, while still obtaining the desired outcome. Additionally, modifications to the disclosed embodiment and the invention as claimed are possible and within the scope of this disclosed invention.

Those of skill in the art would appreciate that the various illustrations in the specification and drawings described herein can be implemented as electronic hardware, computer software, or combinations of both. To illustrate this interchangeability of hardware and software, various illustrative blocks, modules, elements, components, methods, and algorithms have been described above generally in terms of their functionality. Whether such functionality is implemented as hardware, software, or a combination depends upon the particular application and design constraints imposed on the overall system. Skilled artisans can implement the described functionality in varying ways for each particular application. Various components and blocks can be arranged differently (for example, arranged in a different order, or partitioned in a different way) all without departing from the scope of the subject technology.

Furthermore, an implementation of the communication protocol can be realized in a centralized fashion in one computer system, or in a distributed fashion where different elements are spread across several interconnected computer systems. Any kind of computer system, or other apparatus adapted for carrying out the methods described herein, is suited to perform the functions described herein.

A typical combination of hardware and software could be a general purpose computer system with a computer program that, when being loaded and executed, controls the computer system such that it carries out the methods described herein. The methods for the communications protocol can also be embedded in a non-transitory computer-readable medium or computer program product, which includes all the features enabling the implementation of the methods described herein, and which, when loaded in a computer system is able to carry out these methods. Input to any part of the disclosed systems and methods is not limited to a text input interface. For example, they can work with any form of user input including text and speech.

Computer program or application in the present context means any expression, in any language, code or notation, of a set of instructions intended to cause a system having an information processing capability to perform a particular function either directly or after either or both of the following a) conversion to another language, code or notation; b) reproduction in a different material form. Significantly, this communications protocol can be embodied in other specific forms without departing from the spirit or essential attributes thereof, and accordingly, reference should be had to the following claims, rather than to the foregoing specification, as indicating the scope of the invention.

The communications protocol has been described in detail with specific reference to these illustrated embodiments. It will be apparent, however, that various modifications and changes can be made within the spirit and scope of the disclosure as described in the foregoing specification, and such modifications and changes are to be considered equivalents and part of this disclosure.

It is to be understood that the disclosed subject matter is not limited in its application to the details of construction and to the arrangements of the components set forth in the following description or illustrated in the drawings. The disclosed subject matter is capable of other embodiments and of being practiced and carried out in various ways. Also, it is to be understood that the phraseology and terminology employed herein are for the purpose of description and should not be regarded as limiting.

As such, those skilled in the art will appreciate that the conception, upon which this disclosure is based, may readily be utilized as a basis for the designing of other structures, systems, methods and media for carrying out the several purposes of the disclosed subject matter. It is important, therefore, that the claims be regarded as including such equivalent constructions insofar as they do not depart from the spirit and scope of the disclosed subject matter.

It will be appreciated that while one or more particular materials or steps have been shown and described for purposes of explanation, the materials or steps may be varied in certain respects, or materials or steps may be combined, while still obtaining the desired outcome. Additionally, modifications to the disclosed embodiment and the invention as claimed are possible and within the scope of this disclosed invention.

1. A method comprising
receiving a plurality of biological sequence datasets, wherein each of the biological sequence datasets comprises a plurality of biological sequences;
identifying a plurality of combinations of biological sequences, wherein each combination comprises one of the plurality of biological sequences from each of the biological datasets; and
for each combination of biological sequences:
generating a plurality of n-mers for each biological sequence of the combination using a sliding window with length n,
comparing the plurality of n-mers for each biological sequence of the combination with the plurality of n-mers for the other biological sequences of the combination,
identifying distinctive n-mers for each biological sequence of the combination which are not present among the plurality of n-mers for the other biological sequences of the combination, and
determining a number of distinctive n-mers for at least one biological sequence of the combination.

2. The method of claim 1, wherein the plurality of biological sequence datasets comprise a plurality of genome datasets and each of the plurality of genome datasets comprises a plurality of polynucleotide sequences.

3. The method of claim 1, wherein the plurality of biological sequence datasets comprise a plurality of protein sequence datasets and each of the plurality of protein sequence datasets comprises a plurality of protein sequences.

4. The method of claim 1, wherein each biological sequence of the combination is aligned to a reference sequence before generating a plurality of n-mers for each biological sequence of the combination and comparing the plurality of n-mers for each biological sequence of the combination comprises comparing n-mers at the same position of each biological sequence of the combination.

5. The method of claim 1, wherein comparing the plurality of n-mers for each biological sequence of the combination comprises comparing n-mers regardless of the position of each n-mer in each biological sequence of the combination

6. The method of claim 1, wherein determining the number of distinctive n-mers for at least one biological sequence of the combination comprises determining a number of distinctive n-mers for each biological sequence of the combination; and
wherein the method further comprises:
generating a distribution for each of the plurality of biological sequence datasets of the number of distinctive n-mers for each biological sequence of each of the combinations.

7. The method of claim 6, wherein a divergence between each distribution is calculated using one or more of Cohen's D and J-S Divergence.

8. The method of claim 1, wherein each combination of biological sequences comprises a first biological sequence from a first biological sequence dataset and one of the plurality of biological sequences from a second biological sequence dataset, and wherein determining the number of distinctive n-mers for at least one biological sequence of the combination comprises determining a number of distinctive n-mers for the first biological sequence that are not present among the plurality of n-mers for the biological sequence from the second biological sequence dataset of the combination; and wherein the method further comprises:
determining a sequence distinctiveness for the first biological sequence by summing the number of distinctive n-mers from all combinations and dividing by the number of combinations.

9. The method of claim 1, wherein the plurality of biological sequence datasets comprise a first biological sequence dataset and a second biological sequence dataset, and the method further comprises calculating a sequence distinctiveness for one or more biological sequences of the first biological sequence dataset relative to the second biological sequence dataset.

10. The method of claim 1, wherein one of the plurality of biological sequence datasets is a new viral variant sequence dataset.

11. The method of claim 1, wherein n is 9.

12. The method of claim 4, wherein n is 1.

13. The method of claim 1, wherein n is 9-30.

14. The method of claim 1, wherein n is 3-10.

15. The method of claim 1, further comprising identifying common n-mers that are present among the plurality of n-mers for two or more biological sequences in a combination of biological sequences.

16. The method of claim 1, wherein each of the plurality of biological sequence datasets is from a different time window.

17. The method of claim 1, wherein each of the plurality of biological sequence datasets is from a different geographical location.

18. The method of claim 1, wherein each of the plurality of biological sequence datasets is from a different variant.

19. The method of claim 1, wherein the plurality of biological sequence datasets comprises a biological sequence dataset from an infectious agent and a biological sequence datasets from a host organism of the infectious agent.

20. The method of claim 1, wherein generating the plurality of n-mers comprises generating a plurality of n-mers from only a functionally relevant portion of the plurality of biological sequences.

21. The method of claim 1, further comprising using the number of distinctive n-mers or a parameter derived therefrom to predict changes in prevalence.

22. A system comprising:
a non-transitory memory; and
one or more hardware processors configured to read instructions from the non-transitory memory that, when executed cause one or more of the hardware processors to perform operations comprising:
receiving a plurality of biological sequence datasets, wherein each of the biological sequence datasets comprises a plurality of biological sequences;
identifying a plurality of combinations of biological sequences, wherein each combination comprises one of the plurality of biological sequences from each of the biological datasets; and
for each combination of biological sequences:
generating a plurality of n-mers for each biological sequence of the combination using a sliding window with length n,
comparing the plurality of n-mers for each biological sequence of the combination with the plurality of n-mers for the other biological sequences of the combination,
identifying distinctive n-mers for each biological sequence of the combination which are not present among the plurality of n-mers for the other biological sequences of the combination, and
determining a number of distinctive n-mers for at least one biological sequence of the combination.

23. The system of claim 22, wherein determining the number of distinctive n-mers for at least one biological sequence of the combination comprises determining a number of distinctive n-mers for each biological sequence of the combination; and
wherein the operations further comprise:
generating a distribution for each of the plurality of biological sequence datasets of the number of distinctive n-mers for each biological sequence of each of the combinations.

24. The system of claim 22, wherein each combination of biological sequences comprises a first biological sequence from a first biological sequence dataset and one of the plurality of biological sequences from a second biological sequence dataset, and wherein determining the number of distinctive n-mers for at least one biological sequence of the combination comprises determining a number of distinctive n-mers for the first biological sequence that are not present among the plurality of n-mers for the biological sequence from the second biological sequence dataset of the combination; and wherein the operations further comprise:
determining a sequence distinctiveness for the first biological sequence by summing the number of distinctive n-mers from all combinations and dividing by the number of combinations.

25. The system of claim 22, wherein n is 9-30.

26. The system of claim 22, wherein n is 3-10.

27. The system of claim 22, wherein the operations further comprise identifying common n-mers that are present among the plurality of n-mers for two or more biological sequences in a combination of biological sequences.

28. The system of claim 22, wherein each of the plurality of biological sequence datasets is from a different time window.

29. The system of claim 22, wherein each of the plurality of biological sequence datasets is from a different geographical location.

30. The system of claim 22, wherein each of the plurality of biological sequence datasets is from a different variant.

31. A non-transitory computer-readable medium storing instructions that, when executed by one or more hardware processors, cause the one or more hardware processors to perform operations comprising:
receiving a plurality of biological sequence datasets, wherein each of the biological sequence datasets comprises a plurality of biological sequences;
identifying a plurality of combinations of biological sequences, wherein each combination comprises one of the plurality of biological sequences from each of the biological datasets; and
for each combination of biological sequences:
generating a plurality of n-mers for each biological sequence of the combination using a sliding window with length n,
comparing the plurality of n-mers for each biological sequence of the combination with the plurality of n-mers for the other biological sequences of the combination,
identifying distinctive n-mers for each biological sequence of the combination which are not present among the plurality of n-mers for the other biological sequences of the combination, and
determining a number of distinctive n-mers for at least one biological sequence of the combination.

32. The non-transitory computer-readable medium of claim 31, wherein determining the number of distinctive n-mers for at least one biological sequence of the combination comprises determining a number of distinctive n-mers for each biological sequence of the combination; and
wherein the operations further comprise:
generating a distribution for each of the plurality of biological sequence datasets of the number of distinctive n-mers for each biological sequence of each of the combinations.

33. The non-transitory computer-readable medium of claim 31, wherein each combination of biological sequences comprises a first biological sequence from a first biological sequence dataset and one of the plurality of biological sequences from a second biological sequence dataset, and wherein determining the number of distinctive n-mers for at least one biological sequence of the combination comprises determining a number of distinctive n-mers for the first biological sequence that are not present among the plurality of n-mers for the biological sequence from the second biological sequence dataset of the combination; and wherein the operations further comprise:
determining a sequence distinctiveness for the first biological sequence by summing the number of distinctive n-mers from all combinations and dividing by the number of combinations.

34. The non-transitory computer-readable medium of claim 31, wherein n is 9-30.

35. The non-transitory computer-readable medium of claim 31, wherein n is 3-10.

36. The non-transitory computer-readable medium of claim 31, wherein the operations further comprise identifying common n-mers that are present among the plurality of n-mers for two or more biological sequences in a combination of biological sequences.

37. The non-transitory computer-readable medium of claim 31, wherein each of the plurality of biological sequence datasets is from a different time window.

38. The non-transitory computer-readable medium of claim 31, wherein each of the plurality of biological sequence datasets is from a different geographical location.

39. The non-transitory computer-readable medium of claim 31, wherein each of the plurality of biological sequence datasets is from a different variant.
